(19) United States
(12) Patent Application Publication
(10) Pub. No.: US 2004/0005349 A1
(43) Pub. Date: Jan. 8, 2004

(54) Opto-thermal material modification

(76) Inventor: Joseph Neev, Lake Forest, CA (US)

Correspondence Address: SNELL & WILMER LLP, 1920 MAIN STREET, SUITE 1200, IRVINE, CA, 92614-7230, US

(21) Appl. No.: 09/854,311

(22) Filed: May 10, 2001

Related U.S. Application Data
(60) Provisional application No. 60/203,647, filed on May 12, 2000.
(60) Provisional application No. 60/215,657, filed on Jul. 1, 2000.
(60) Provisional application No. 60/237,548, filed on Sep. 30, 2000.
(60) Provisional application No. 60/272,653, filed on Feb. 28, 2001.

(51) Int. Cl. A61K 9/70; A61N 1/30

(52) U.S. Cl. 424/443; 604/20

(57) ABSTRACT

A device and a method for removing or modifying a material substance, comprising: applying energy to an intermediate material, said intermediate material subsequently delivering said energy to the target material. The device and the method also utilize active energy removal schemes in order to minimize pain, and/or protect some tissue and or control tissue modification and removal effects.

A system for enhancing and improving the delivery of a substance across a barrier. A substance is applied to the surface of the barrier to be penetrated. The substance is then brought in contact with a device containing an energy source, said energy source emit energy which causes an intermediate medium to drive the substance across the barrier.

### PRIORITY CLAIM

This patent application claims the benefit of the filing date of U.S. Provisional Patent Application Serial No. 60/203,647, filed on May 12, 2000 and entitled APPLICATION TECHNOLOGIES, the entire contents of which are hereby expressly incorporated by reference.

### BACKGROUND OF THE INVENTION

Recent studies of tissue healing process have demonstrated that an injury to the upper layers of a person skin will result in collagen regeneration and the growth of a more elastic, younger looking skin.

Several methods for generating this effect have been attempted. A mild photo-damage will cause some blistering followed by a natural removal of a few outer layers of the skin. Such a process usually results in a younger looking skin. Similar results can be obtained from a mechanical or opto-mechanical removal of a few surface layers by an abrasive or ablative processes. One such process is known as micro-dermabarasion and involves the removal of skin by a stream of Aluminum oxide particles aimed at the surface of the skin. Alternatively, a mechanical scraping of the skin outer surface layers with an abrasive material such as fine sand paper is also deployed to achieve such abrasive effects. A more sophisticate yet expensive method involves Er:YAG laser sources of relatively short pulse duration (for example a few hundreds microseconds long) and a highly water-absorbed wavelength of 2.94 micrometer (Or, alternatively 2.79 micrometer of the Er:YSGG free-running lasers). Other methods involve somewhat longer exposure to somewhat more deeply water-penetrating beams such as those of the 9.6 micrometer and 10.64 micrometer CO₂ laser beams to generate a layer of thermally damaged surface a few hundred micrometer thick. Such deeper skin tissue coagulation usually results in the most aggressive tissue damage and the longest healing time but also in the most effective removal of wrinkles and most effective “Skin rejuvenation” and younger-appearing skin.

These prior art procedures represent some beneficial results but also provide potential risk to the patients in the form of excessive damage and danger of scarring. Abrasive processes often result in excessive cutting, bleeding and pain and sometimes lead to infection, and scarring. Laser treatments are expensive and often result in significant pain excessive thermal tissue damage and lead to permanent scarring.

### SUMMARY OF THE INVENTION

The present invention provides a method and apparatus for the removal and modification of target material and in particular the removal and modification of the human skin and underlying tissue layers.

A substance which absorbs electromagnetic radiation well in at least a portion of the incident beam electromagnetic spectrum is applied to the surface of the target material. The target material is exposed to electromagnetic radiation, which includes the portion of the spectrum that is absorbed by the substance applied to the surface. At least some of the absorbed energy of the electromagnetic radiation source is absorbed by the substance of high absorption and is converted thermal energy at the surface of the target material. The thermal energy thus generated is then responsible for vaporizing and ablating part of the skin and for irreversibly thermally modifying portion of the target material. A preferred embodiment envisions the use of carbon-based pigments in suspension within a host material and a continuous emitting electromagnetic radiation source.

The device described in this invention allows effective, safe and easy-to-use applications of the high absorbing material to the target material surface for the purpose of converting optical energy into thermal energy and affecting a change in the target material using said thermal energy.

### BRIEF DESCRIPTION OF THE DRAWINGS

Preferred embodiments of the present invention can be described by reference to the drawings.

FIG. 1 is a cross-sectional representation of human skin with a substance of high absorption applied in the form of a cream or lotion.

FIG. 2 is a cross-sectional representation of human skin with a thin film containing the substance of high absorption, attached to the skin.

FIG. 3 is a schematic representation showing the pattern of application of electromagnetic radiation and a cooling spray.

FIG. 4 illustrates the two zones of target modification including zone of ablative material removal and zone of thermal material modification.

FIG. 5 illustrates the four-parameter regimes that interact together to bring about the desired target modification effect.

FIG. 6 illustrates experimental data showing tissue modification depth as a function of source's power and other relevant parameters.

FIG. 7 illustrates the effect of the density of particles of high absorbing substance on the amount of energy coupled to the target surface.

FIG. 8 illustrates an apparatus for a high absorbing substance film

FIG. 9 illustrates a telescopic attachment for adjusting the distance of the high absorbing substance film from the energy-focusing element.

### DETAILED DESCRIPTION OF THE PREFERRED EMBODIMENTS

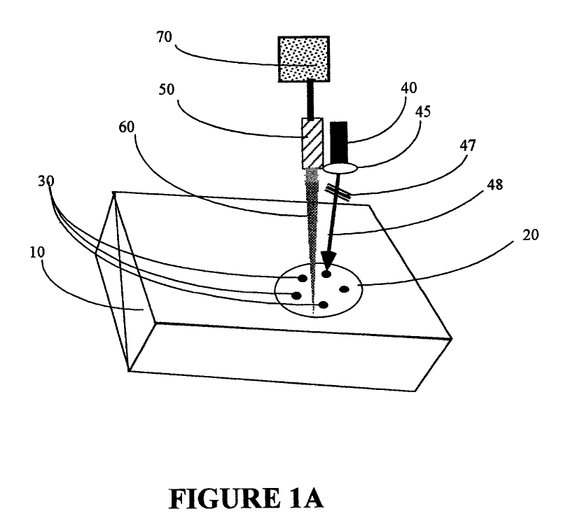

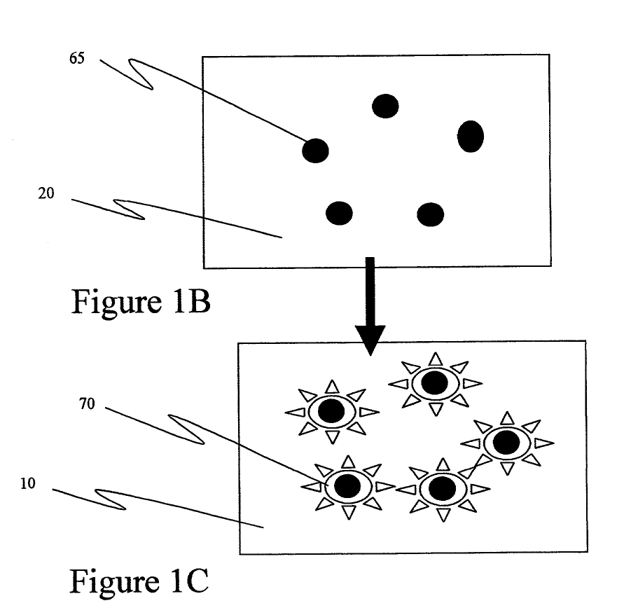

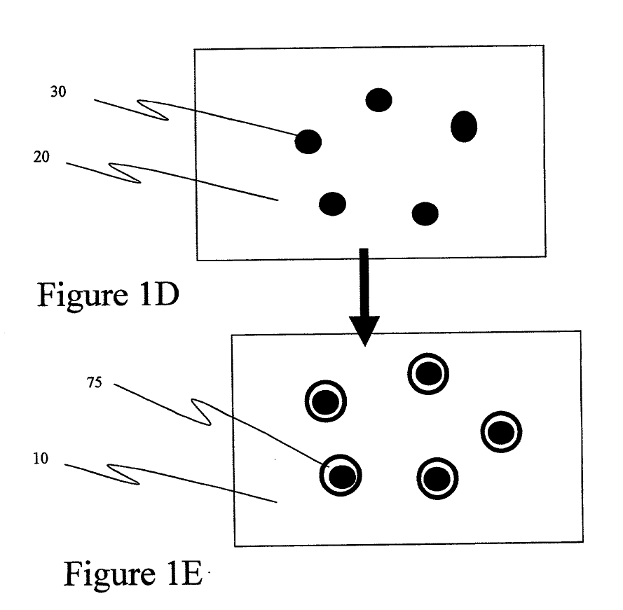

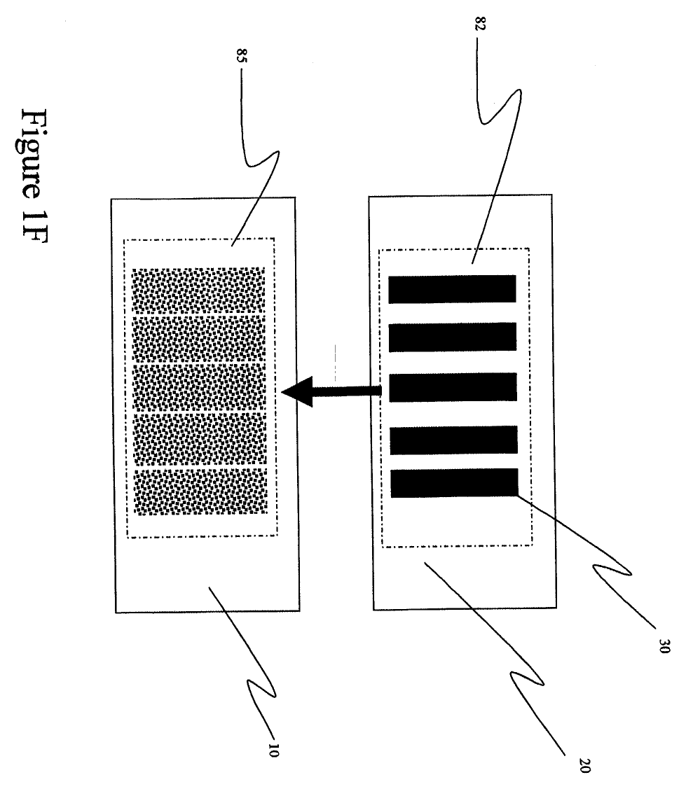

An embodiment of the present invention can be described by reference to FIG. 1. FIG. 1 shows a cross section of a target tissue such as the human skin. A layer of high absorbing substance 10 is placed on top of the epidermis 20 which in turn is overlying the dermis 30 and fat 40 layers. The layer of high absorbing substance is contained within a lotion or liquid which is applied to the target material. This method has the disadvantage of being harder to apply and of accidentally contaminating other location on the target material. On the other hand, the method is simple because the high absorbing substance is at least partly in direct contact with the target material thus leading to the most efficient transfer of the optically absorbed thermal energy to the tissue.

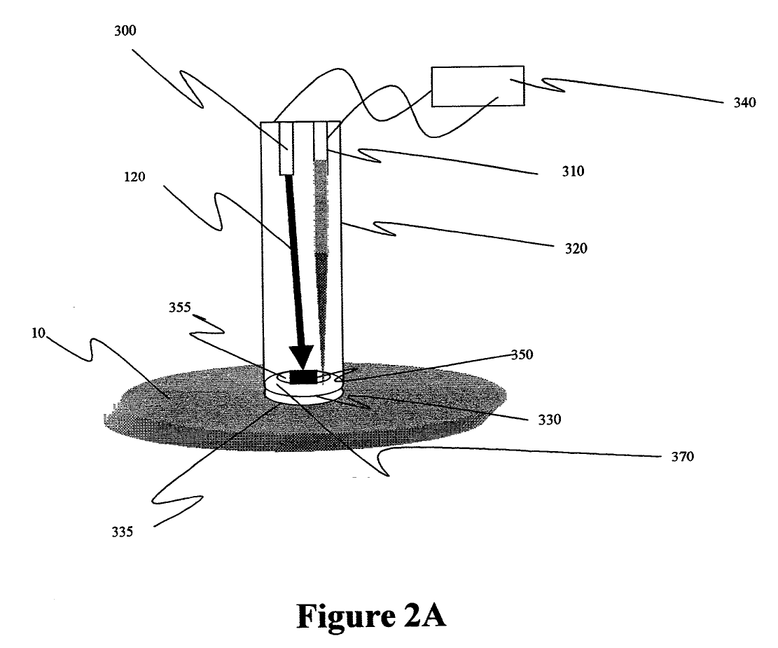

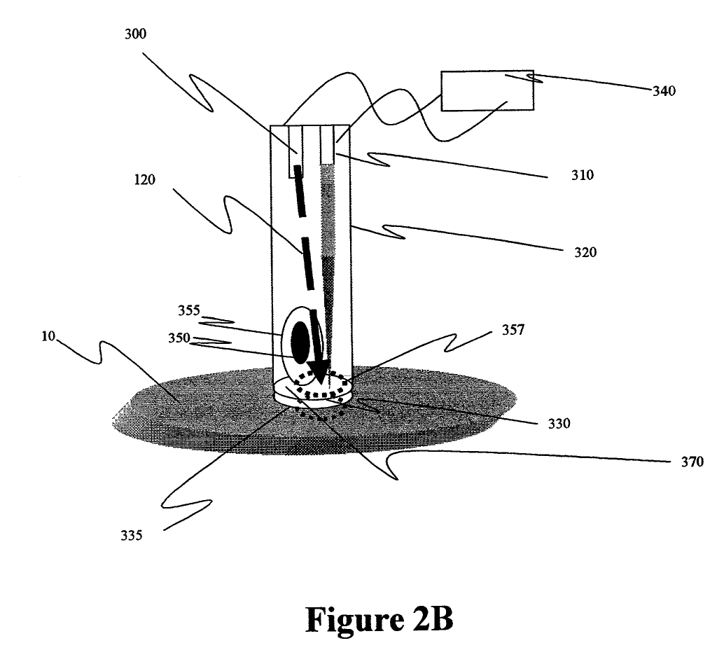

FIG. 2 shows a preferred embodiment of the invention. A layer of high absorbing substance is now placed in the form of thin film 100 containing particles of the high absorbing substance, on top of the epidermis 20. The epidermis 20 in turn is overlying the dermis 30 and fat 40 layers. The layer of high absorbing substance is contained within a lotion or liquid, which is applied to the target material. This method has the disadvantage whereby is a cross-sectional representation of human skin with a thin film containing the substance of high absorption, attached to the skin.

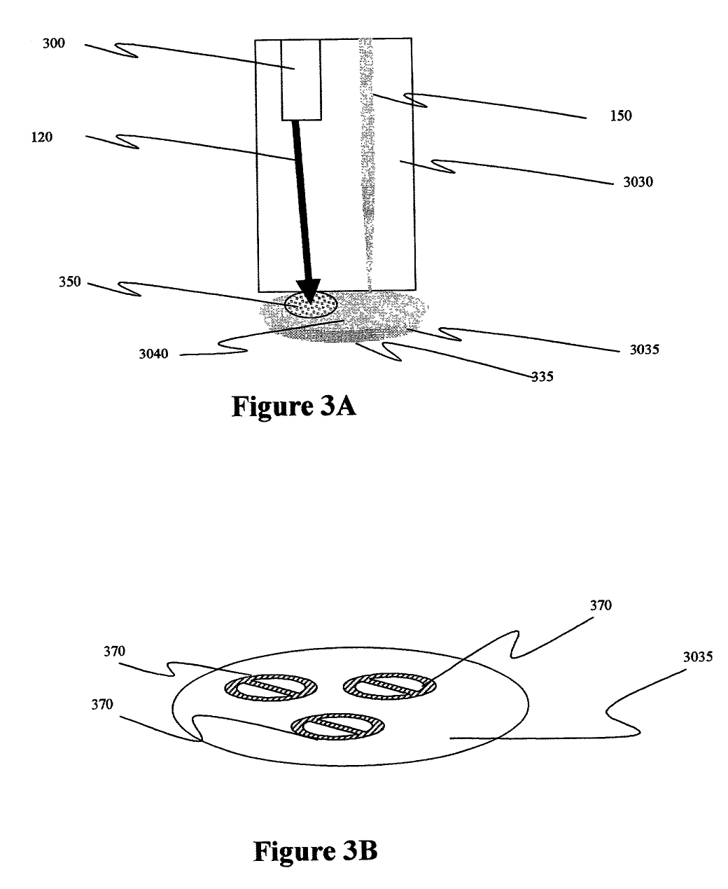

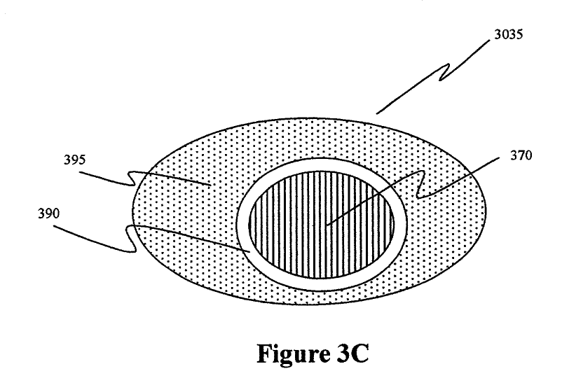

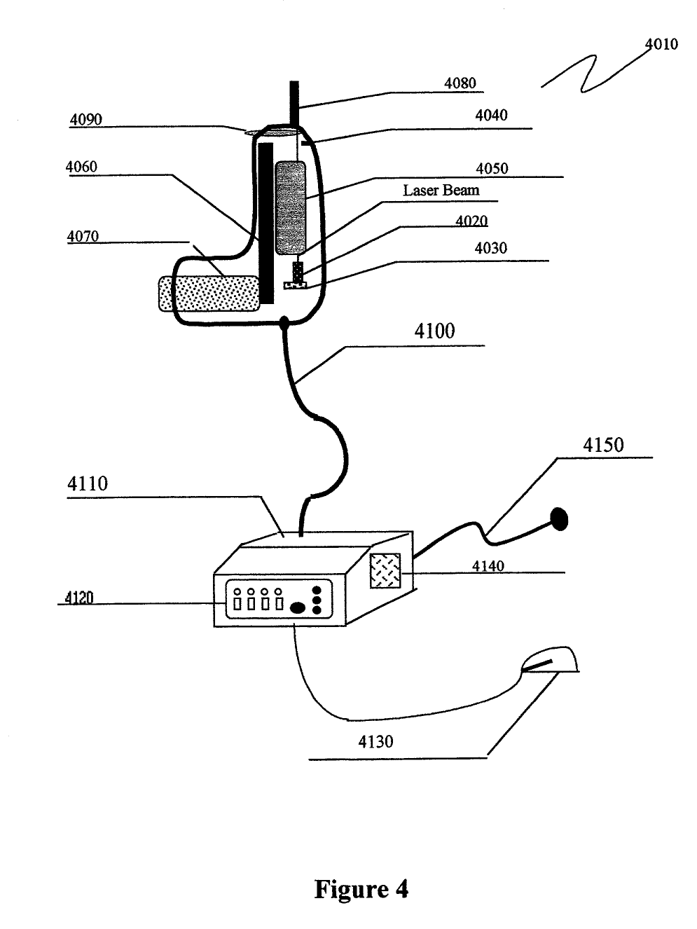

As FIG. 3 shows, a light source 130 emits energy 120 towards the high absorbing substance 10. The high absorbing substance 10 is capable of absorbing at least some of the energy 120. The energy is subsequently thermally conducted towards the deeper portion of the epidermis 20, the dermis 30 and the deeper fat layer 40. As FIG. 4 shows if sufficient energy is deposited in the tissue, an ablative zone, 300, where target material has been physically removed, is formed at the outer target layer. In the case of a human skin portion of the epidermis, and, in case of more aggressive interaction, the entire epidermis and even parts of the dermis, may be ablatively removed. Further thermal diffusion and deposition of energy will also lead to the formation of a zone of irreversible damage, 320. Here target material has been thermally altered but not physically removed or ablated. In the specific case of human skin, the tissue in the zone 320 will usually be thermally coagulated.

Such thermal coagulation and thermal injury to the skin tissue will lead to collagen regeneration and the appearance of younger looking skin.

Topically applying a thin layer of high absorbing substance such as carbon particles, Indochina green, graphite, black food coloring, china ink, acrylic tattoo inks, and black pigments among others, will result in an efficient conversion of electromagnetic source energy to thermal energy.

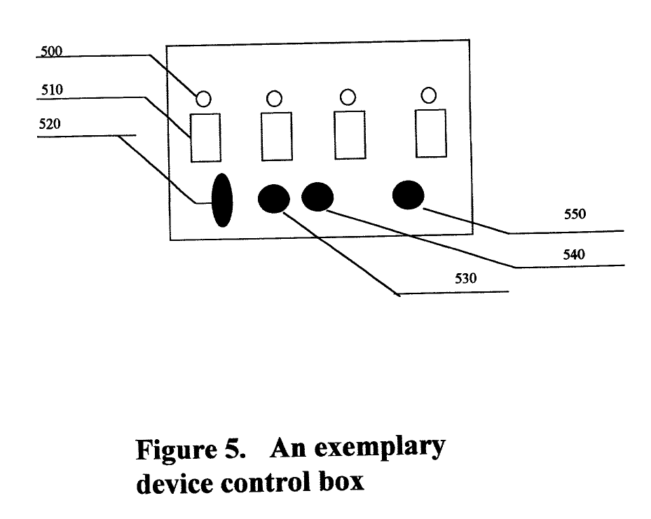

As is shown in FIG. 5, the conversion efficiency (i.e. how much of the incident energy has been converted to thermal energy) depends strongly on the interaction between four factors: The energy source parameters (Lp) 500, the energy manipulating components parameters (OS p) 510, the Energy removing parameters (ER p) 520 and the high absorbing substance parameters (HASₚ) 530. The present invention contemplates four interacting parameter regimes that yield to the final target modification effect. The first parameter group to be considered 500, is the source parameter. These include the source energy, the source power, the pulse duration (if pulsed) and Pulse repetion rate (if pulsed), and the source wavelength, if the enrgy is radiated.

The second 510 group of parmaters is the optical/scanning parameters, These define the beam spot size and the motion of the energy along the intermediate material

The third group 520 is intermediate material paramters. These define the rate of conversion of energy from the source enrgy to the type of energy that interacts with the target. The final group of parameters 530 is the energy removal paramteres. These define when, how and temporal nature of the energy removal from the intermediate material, target material or both. The interaction of these paramters amongst themslvers is illustrated schematically in FIG. 5.

The interaction between these four components, illustrated symbolically in FIG. 5 by the circle 540, will determine how much of the source's energy is converted to thermal energy.

For example, I have tested a 1 W source scanning a 1 Cm² area of at a rate of about 6 seconds. Assuming that all the energy is absorbed by the layer of high absorbing substance, we have 6 Joule deposited over the 1 Cm² area.

Assume now that the energy absorbed E is thermally conducted to the underlying target area. In the case of human skin, for example, water molecules dominate the underlying tissue cells. Simplifying our analysis by assuming as a first approximation a tissue model which is similar to an equivalent volume of water, we can estimate the amount of energy it would require to take a coagulate (i.e. bring the tissue temperature to above the temperature of thermal denaturation of approximately 60° C.) a volume of tissue (100 μm*1 cm²) approximated as a volume of water.

We can use the relationship

E=CΔT (1)

Where E is the energy required and AT is the increase in temperature.

To take water from 20 degree c to 60 degree c and with the specific heat of water given by approximately Cs=1 cal/g*c⁰

We get from the relation above E˜1.6J

To vaporize this amount of tissue would require bringing the tissue (or in our simplified thermal model—the water volume, 1 cm² 100 μm thick) to boiling temperature, and then, further vaporizing that volume. From the relationship of Equation 1 above, the temperature rise of 80° C., would require about 3.2 J. The heat of vaporization of water at 100° C. and 1 atmosphere pressure is 539.6 Cal/gm. Thus to vaporize the 100 um thick volume discussed above will require bout 21.6J, clearly much more than the TOTAL incident source energy in our experiment of about 6J. On the other hand, a 10 mm thick layer of tissue (approximated as water) will require only approximately 0.32 J to raise its temperature from 20° C. to 100° C., and 2.16 J to vaporize. With our above calculations show that to raise an addition volume of (100 μm×1 cm²) to 60° C. coagulation temperature is about 1.6 J, we see that with a total of about (2.16 J+0.32 J+1.68 J=4.16 J we can vaporize about 10 μm of tissue and coagulate an additional 100 μm thick layer.

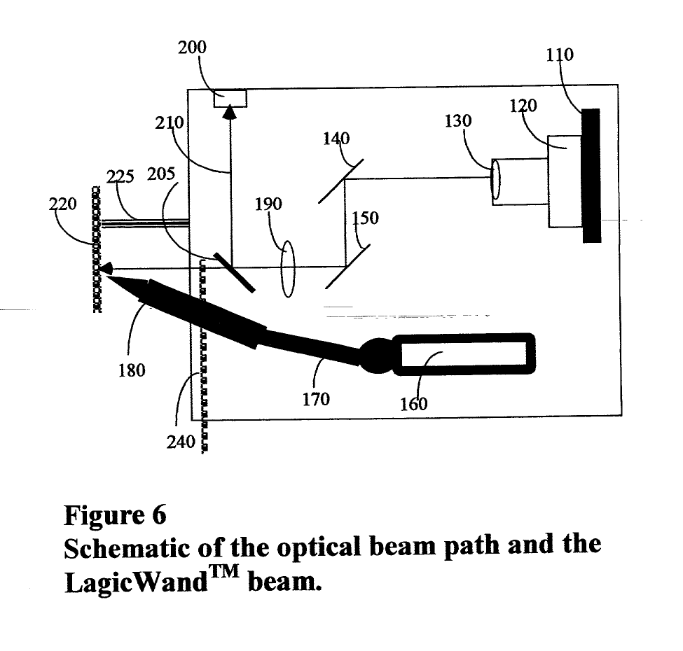

This simplified analysis agrees with the order of magnitude of our experiments as illustrated in FIG. 6. The experimental results of FIG. 6 shows the depth of tissue ablated 620 and thermally damaged 630 in pig skin tissue. The figure shows ablation and thermal damage depth on the tissue depth dimension axis 610, as a function of laser diode fluence, 600, (power=1 W, scan rate is 1 cm²/sec, and wavelength=810 nm absorbed by a high absorbing substance composed of carbon suspension).

We can clearly see that both the depth of ablation and depth of tissue thermal damage are on the order of magnitude predicted by the above analysis.

Preferred Embodiment—Controlling Tissue Effects:

1) From the above analysis we can conclude that coupling of X Joule of energy with temporal T(t) and spatially R(r) of energy will lead to the ablation of a layer of thinness Xabl and leave behind a layer of thickness Xtd of thermally modified tissue. The present invention contemplate four parameter groups responsible for a given energy distribution

E(t,r)=F[T(t)R(r)]

The four parameter group are illustrated schematically in FIG. 5. These include the laser parameters, 500, the beam-modifier parameters (including the optical beam modification apparatus and scanning parameters) 510, the Energy Removal parameters, 520 and the high absorbing substance parameters, 530.

The example above showed that a given parameter combination with 1 W of 810 nm radiation, allows, for example, the generation of 10 um deep ablation zone and a subsequent thermal modification zone of approximately 100 um deep. Such an exemplary combination generated 6 J of energy, of which, according to the above analysis, about 4.2 J was needed to generated these effect. If we define this energy as Eth, or threshold energy for interaction, which when applied in conjunction with the parameter combination 540 of FIG. 5, will result in the Zabl=10 um and Ztd=100 um.

Clearly, when considering the parameter involved in the substance of high absorption (HAS), 530, if a single layer of absorbing particles is involved, the surface density of absorber will correspond to the amount of energy coupled to the substance of high absorption and the total amount of incident energy converted into thermal energy. For the parameters in the incident power 1W, 810 nm, with scan rate of 1 cm² per six seconds, described above, a single layer HAS with particle density such that only, for example, ⅓ of the target area is cover, will not provide sufficient energy to achieve the effect of vaporizing 10 um of tissue and thermally modifying 100 um. While thermal diffusion will provide thermal effect even through the gaps in the surface coverage, (for example, if a heat-removing substance is applied every 250 ms, heat diffusion of about 500 um within this time, would essentially assure complete coverage of the target surface by energy) the total amount of energy deposited in the tissue will be ⅔ less then in a uniformly covered surface as the portion of the beam that does not encounter HAS particles, continues to propagate through the skin unimpeded.

2) On the other hand, if E>Eth, then one can compensate for the high absorbing substance, HAS, lower particle density, with a higher source power.

For example, if in the experiment discussed above we employ a power source of, for example, 3 W, the sources now deliver 18J in 6 sec, and while ⅓ of the particles will couple approximately ⅓ of the energy, the same total quantity of energy will be coupled to the targeted. Heat diffusion will then assure that this same overall quantity of coupled energy will be distributed to the entire area.

With a spot size of, for example, about 200 um and a dwell time of about 10 ms, even small, roughly single micron size particles, will allow heat diffusion to roughly the same area on the same time scale. Thus a uniform HAS particle coat with a 1W beam dwell time of about 10 ms will cover about he area with thermal energy as a coat with ⅓ the particle density but with a 3 W power.

The above discussion thus demonstrate that the parameters combination 540 of FIG. 6, allow compensation of sources power by varying the High absorbing substance concentration and vice versa.

3) The thickness of the high absorbing substance is another components of the parameter group 530 of FIG. 6 that plays an important part in determining the target (or tissue) effects. If the absorbing particles are poor thermal conductors, depositing a multiplayer absorbers on top of the target will result in significant source's energy being absorbed in the upper layers but never making it to the lower layers before the heat remover is applied. ON the other hand, applying a high absorbing particles in suspension in a substance with improved thermal conductivity will assure that the above energy is conducted to the target material. One can then allow gradual thermal energy application to the target as sequential scan passes are stacked and add their thermal energy to the tissue. The present invention contemplates ablatively removing portion of the high absorbing substance coat with each pass resulting in a self-limiting scheme allowing only a finite number of passes and thus only a finite amount of energy deposited in the target material before the high absorbing coat has been completely removed and no more sources energy can be coupled to the target.

4) Controlling Tissue Effects Through the Heat-Removal Mechanism.

The energy removal mechanism 520, illustrated in FIG. 5 also plays a multiple role in controlling the tissue effect. As was pointed in the discussion above, it effectively ends a heating cycle by effectively removing the heat from the outer surface of a target. For example, if a single line 1 cm long and 0.7 mm wide is scanned in, for example, 100 ms, then heat diffuses about 300 um from the impacted zone. Activation of the heat-removing mechanism (according to the pattern shown, for example, in FIG. 3) allows control of spatial distribution and synchronized termination of the thermal effect (by effectively removing the heat source from the surface). In fact, the action of the heat removal mechanism goes even further, because by allowing the operator to very rapidly readjust the surface temperature to even below normal ambient temperature, heat can now be forced to flow out of deeper layers in the tissue. The net effect is creating a spatially and temporally controlled thermal pulse that propagates into the material bulk from the high absorbing substance in contact with the material surface layer and then, upon instruction from the operator, the heat reverses direction and flows out of the bulk.

An analogy to the behavior of the flow of thermal energy in the material and the time and space dependence of the distribution of thermal energy density (thermal energy per unit volume) is the behavior of an electric charge density (for example electrons density) under the influence of alternating positive and negative electrodes outside a conducting medium. The thermal energy density under the influence of the deposited light energy (effect of 530 in FIG. 5) and the heat removing mechanism, 520, (such as freon like spray or other cryogen spray or controlled air flow, or temporary contact with cold plates or thermoelectric coolers or other such methods to induce transient heat removal), is much like an oscillating or direction-reversing electron cloud movement.

The following are additional preferred embodiments (PE) contemplated by the present invention. The discussion below will also demonstrate the relationship between the various parameter groups of FIG. 5.

embodiment 1: Optical heating, no high absorbing substance and no heat removal mechanism is used.—heat transfer amounts substantially to diffusion In this case. Initial thermal energy distribution mirrors that of the optical deposition. Heating due to optical scattering is maximal just below the surface.

Emobdiment 2. Heating with a heat removal applied before and or during irradiation, resulting in peak power and thermal energy distribution below the surface.

Embodiment 3: Heating the target surface optically resulting in initial thermal distribution which mirror the initial optical distribution, Heat removal is then applied to the surface resulting in modification of thermal distribution below the surface as well as heat begins to redistribute itself and diffuse BACK UPWARD towards the now cooler surface. Thermal modification thus does not allow enough time for surface to get fully heated and damaged. Thermal distribution below the surface is temporally and spatially modified according to intended operator design.

Embodiment 4: Application of a highly absorbing substances to the target surface prior to external energy deposition. This leads to initial heating confined to the THICKNESS OF SURFACE high absorbing substance alone. The operator thus has full control over the deposition layer and the initial heating zone.

Prefered Embodiment 5: Initial heating with high absorbing substance allowing heat to propagate ahead a predetermined distance. A heat removal mechanism is then applied to the surface quenching the surface heating. The heat then continues to diffuse deeper into the tissue but the thermal energy below the surface also flows back out and flows back towards surface where it is used to reheat surface. The net effect of this process is to limit the effect of the heating, the amount of heat available for tissue modification and the extent of the damaged tissue and the depth location of damaged tissue.

Preferred embodiment 6: The high absorbing substance is applied and subsequently wiped off so it remains substantially mostly in the pores, depressions and troughs of the skin. In these location it absorbs radiation causing localized points of thermal heating spatially distributed on the target surface. Subsequent illumination but the external energy source, results in some of the radiation being absorbed by the localized points containing high absorption substance while the rest of the radiation propagates deeper and heating—to a much lower degree—much deeper into the tissue.

The net effect is rapid heating and expansion of the pores COMBINED with heating of lower/deeper region of the skin to allow expansion of the lower parts which, in turn, expel through the pores, undesired material which may reside in the skin.

Preferred embodiment 7: The preferred embodiment above (PE6) followed by the removal of heat. “Freezes” and the expansion on the surface (and begins generation of contraction of the target material) while heating of deeper regions by Optical deposition allows an efficient deeper target expansion.

Preferred embodiment 8: The process then of application a high absorbing substance can be modified through a high absorbing substance applicator device to achieve the following effects:

1 Heating of a very thin layer of high absorbing substance. Heat is transferred to the skin.

2 Heating of a “diluted” high absorbing substance suspended in a layer of other material

3 Wherein other material is an insulator—thereby mitigating the amount of heat transferred but also maintaining heat in that layer for a longer time.

4 Wherein the other material is an insulator—thereby mitigating the amount of heat transferred down to the lower tissue but allowing accumulation of heat in the surface layer lowing to material removal through explosive ablation of the applied surface layer.

5 Wherein other material is a conductor—thereby enhancing heat transfer to the tissue.

6 Whereby the above method is followed by surface heat removal.

7 Where the high absorbing substance is also insulating thus resulting in ablation of surface with substantially mostly mechanical shock to tissue.

8 Wherein the high absorbing substance is partially transmitting resulting in BOTH deeper “Optical” heating AND intense surface heating.

9 The device and method of 8 above wherein the heating phase is followed by heat removal resulting in intense heating at below surface but a more delicate spatially larger heating at the surface.

Preferred embodiment—Application Technologies—Application of high absorbing Substance Using a Thin Film:

Another way to create a precise effect with the disposable is to create a film. For example one can create a uniform suspension of high absorbing substance in a host material such as paraffin, paper, metallic matrix, insulator matrix, thermal insulator or plastic matrix, thermally conducting matrix, Jelly, agar or other hosting material. The operator can then slice it to a precise thickness (for example from a preferred thickness of about 10 micrometer to as much as about a 100 micrometer thick—although many other thickness can be contemplated). The process is much like that used in histological slides preparation. A slice with the desired particle size and the desired concentration is prepared and is coated with adhesive to allow it be attached to the targeted area of the skin

Such a film of HAS can be packaged in a sterile packaging and can come in various sizes and shapes. Such a package can then be opened prior to use, and the operator can cut it with sterile scissors to the desired shape. The film is then attached over the desired targeted area creating a precise special localization of concentration and density and particle size and thickness of layers.

Light traveling through the film containing the high absorbing substance, activates the product in order to cause thermal injury to tissue and/or the high absorbing substance itself for the purpose of causing thermal injury to tissue. The solid film become adhesive upon contact with moist target and adheres to a given location. The adhered film allows high spatial control and eliminates the more messy cream or lotion method of applying a substance of high absorption. The film visually changes in color as the high absorbing substance interacts with the incident energy. Some portions of the film are consumed by the applied light source.

The consumption of the film shows the operators where they have treated, and how long and when the product has expired.

With the film or cream of high absorbing substance in place, an site undergoing interaction is visually altered and the handpiece may be used in a free hand motion to cover a larger area. The film will change in appearance, showing the area treated and Confirming the product has been consumed. The operator continues to move the handpiece over the target area until the film changed its appearance uniformly to the one indicating proper treatment.

The film may be designed to be thin enough so it does diminish the effectiveness of thermal energy conduction due to the energy source or heat removing source that are heating or cooling the skin. The film may incorporate anesthetics drugs nutrients and coolants that could allow the film to have it own cooling mechanism built into it.

Preferred Embodiment—

The Device contemplated include an energy source which emit radiation which, in turn, is absorbed by the an intermediate material made of thin layer. One surface of the intermediate layer contains an absorbing substance, which is capable of absorbing the radiation of the energy source inside the handpiece. A second property of the intermediate substance is that it is capable of transmitting said absorbed energy from the side facing the energy source to the side in contact with the target material (the material to be modified). A third property of the intermediate material is that all the absorbing substance is contain in a region of the intermediate material, which is accessible to the radiation from the energy source but which is—in a preferred embodiment—NOT in contact direct contact with the target material.

In an alternative embodiment, the intermediate material contains absorber that IS in contact with the target material. However, said intermediate material and the high absorbing substance of this embodiment are made of biocompatible material which can be used in contact with an animal skin without causing adverse effect. In the more general case, said high absorbing substance and intermediate material can be in conatct with the target material but are made of material which DOES NOT have adverse effect on the target material. For example, High absorbing material can consist of carbon particle in solution as in Higgins Black Magic ink. A paper matrix can be a 0.05 mm thin paper with the carbon solution capable of adhering to the surface of the paper film.

Further preferred embodiments for the application of high absorbing substance:

A) The composition of the high absorption substance can be adjusted for an optimal tissue modification. The matching to optimize the interaction 540 in FIG. 5 is accomplished by adjusting the source parameters P_{L} 500 and Optical/scanner parameters Po/s 510 in FIG. 5, to that of the high absorption substance parameters P_{HAS}, 530.

Settings for Laser/Optical/Scanner—

The fluence F at a given point and time on the target surface is given by,

F=P/(LₓNUₓD)

Where P is the source's power in Watts, Lx is the length of scan in the X-direciton (the horizontal direction), NUₓ is the scan frequency in the X-direction and D is the spot size diameter.

The actual energy in the tissue is determined by a combination of the effect of the energy absorbed by the high absorbing substance and its conduction coefficient (CC) responsible for transferring the thermal energy to the tissue.

Thus, the thermal energy transferred to the tissue (recall that the fluence is simply the energy per unit area) is proportional to

E tissue˜(E absorb in cream)*Conduction coefficient

Now if we use an absorber which also acts as an insulator in the medium, the conduction coefficient then becomes a function of the high absorption substance density (ρ).

E abs=FUN(ρ)

If we design the high absorbing substance (HAS) as a good insulator, material conduction will in general decrease as a function of HAS density ρ.

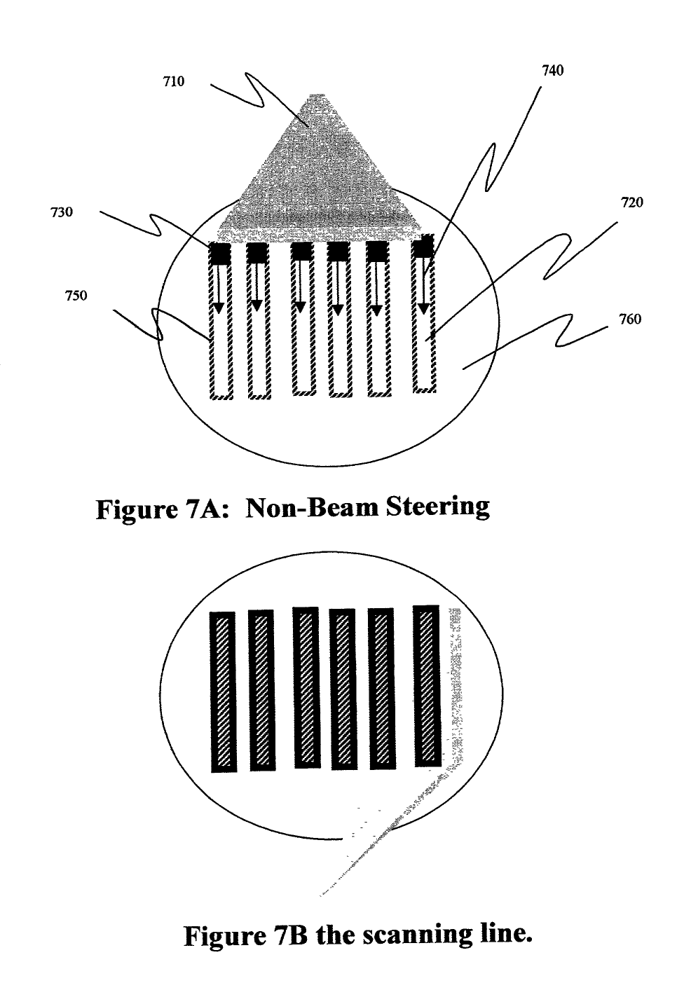

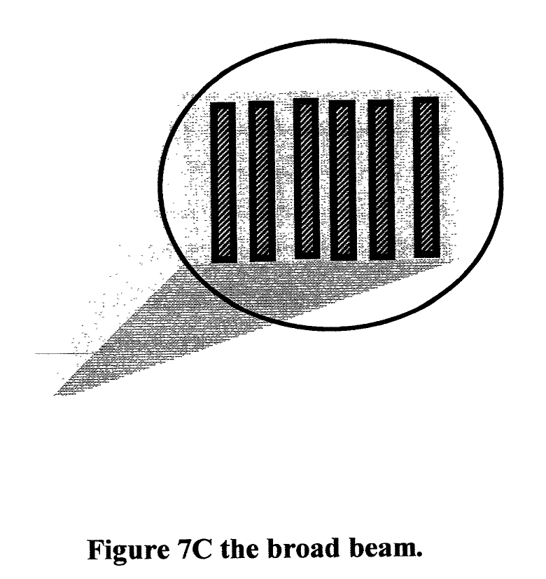

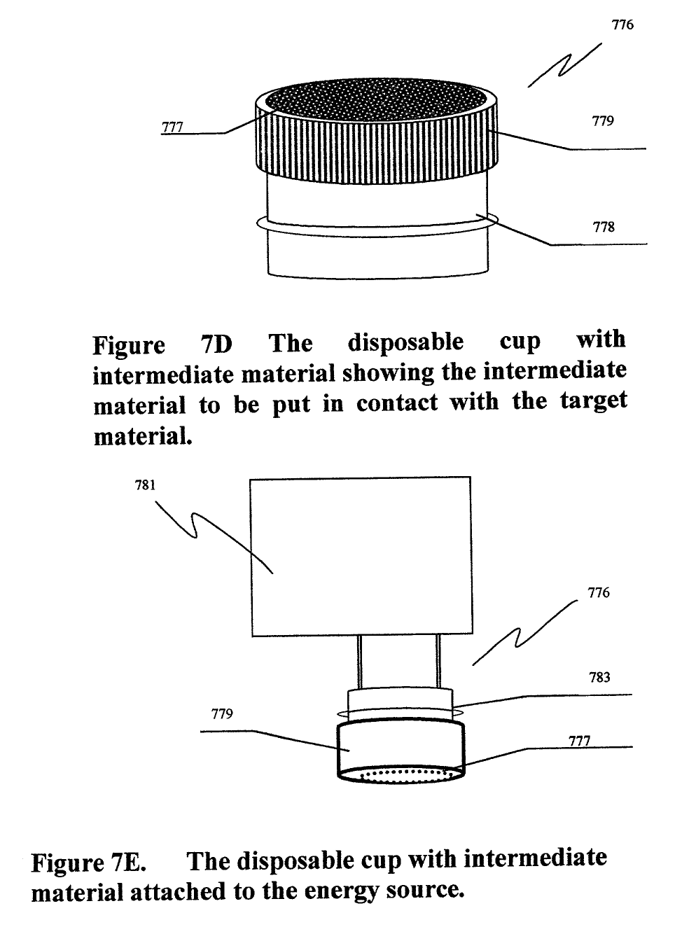

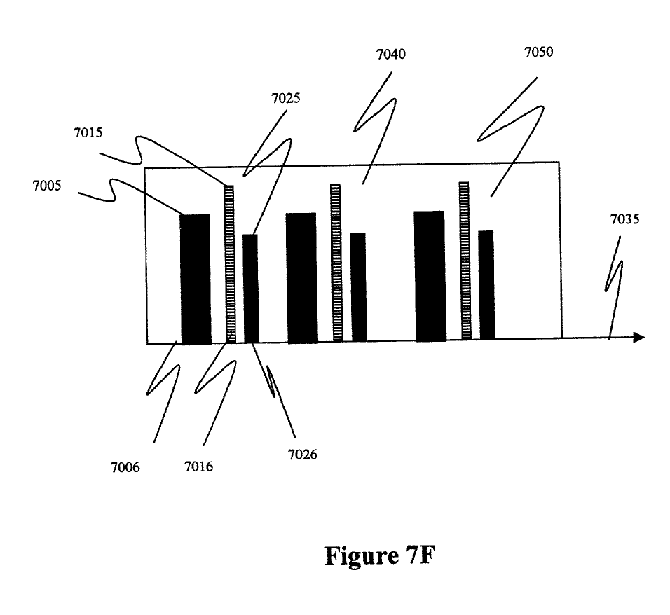

With these considerations we have effectively designed a system such that incident energy conversion to thermal energy is increase with the HAS density BUT the transfer of this thermal energy from the layer of HAS to the targeted material surface is DIMINISHED with increase HAS density. This situation is depicted by FIG. 7 below.

As shown in FIG. 7, the absorbed energy 710 increase with HAS density 700, while the transferred thermal energy to the tissue 720 is decreased as a function of HAS particle density. The total amount of energy transferred to the targeted material surface, 730, is thus, a combination of these two effect and is demonstrated in FIG. 7 by the curve 740. Optimal and maximum thermal energy deposition in the targeted tissue surface is corresponds to the location 750 shown in FIG. 7. The curve 720 shows the tendency to decrease coupling due to increase HAS particle density and the associated decrease thermal conduction. The curve 710 shows the tendency to increase energy coupling with increase HAS particle density and increased absorption. The curve 740 shows the actual effective energy coupling to the target material surface due to the combined effect of curve 710 and 720

The correct calculation of the amount of energy depostitede in the target material should thus be

F_{effective}=P_{effective}/(L NUₓDiamter)

Where F_{effective} is given by

P_{effective}=(Incident power*Absorption)

To calculate a manipulation of the beam power as a function of HAS particle density we follow the procedure below:

We Assume a uniform complete absorption in tissue when ρ=P_{ideal}

We then assume that when F=F_{ideal} one gets the desired tissue effect

We Set laser/optic/scan parameters—Power, Lx, NUx and beam diameter at the target so that to F>>F_{ideal}

So we are certain that a thermal damage to the target material will occur.

We then REDUCE ρ to ρ<ρ_{ideal} so that LESS power is absorbed and the incident fluence is again in the close of the “ideal” fluence F_{ideal}.

Obviously the dependence of HAS particle density on Laser/optical/scanner parameters to achieve a desired tissue or target effect allow a large number of permutation and large number of combinations to be selected.

If a Different high absorbing substance (HAS) particle density ρ is provided and if the HAS particle density is too low—no effective interaction occurs On the other hand, if the HAS particle density is too high—a burn might occur

If HAS particle and the host substance are maintained at a constant conductance level, the deposited energy density will increase monotonically as a function of the HAS particle density until the surface is completely covered with HAS.

Such a situation in combination with the increasing source power levels will result in increased thermal loading and ultimately burn and blister.

Description of Such Preferred Embodiment:

The film is designed to be thin enough so it does not affect the cryogen that is cooling the skin. The film can incorporate anesthetics drugs nutrients and coolants that could allow the film to have it own cooling mechanism built into it.

One way of creating such a film that adheres to the surface is using a starch paper such as rice paper or potato starch paper. High absorption substance particles according to the correct composition proportions and design described above may be incorporated into the starch papers or may be added after the paper has been form. The method allow precise control of the thickness of the film or paper sheet, as well as precise control of the paper composition and HAS particle density, particle size and chemical content. The thin film or paper is then, in a preferred embodiment, being consumed with successive paths so at the end of a pre-determined number of passes, substantially very little HAS particles are left behind and the interaction is self-suspended adding to the safety of the device.

Preparation of a Film Comprsining of a Potato Starch Film with High Absorbing Particles Suspended Within it can be Accoumplished via the Following Procedure:

1.5 cup of water

1 spoon of potato starch

Mix starch in Cold water first

Stir thoroughly and make sure all is dissolved

Place mixture in pot and bring to a boil (½ way)

Mix in HAS into liquid (Food coloring carbon particles)

Bring again to a boil allowing liquid to rise close to the top of the pot (Stir regularly to make sure no clumps form)

Remove/turn off heat and allow to dry on the side of the pot. Approximately 15 min depending on thickness.

If it is too thin redo but at lower temperatures.

It is very important that the liquid and starch concentration determine how thickly the side of the pots will be coated. The process can be repeated a few times to make a thicker coat.

Another way to create a precise effect with the disposable is to create a film by other means. For example one can create a uniform suspension in a paraffin, or in a Jelly or agar. Then slice it to a precise thickness (e.g. 5 micrometer) much like we do in histology slides preparation. A slice with the desired particle size and the desired concentration is prepared and is coated with adhesive to allow it be attached to the targeted area of the skin

Such a film of HAS can be packaged in a sterile packaging and can come in various sizes and shapes. Such a package can then be opened prior to use, and the operator can cut it with sterile scissors to the desired shape. The film is then attached over the desired targeted area creating a precise special localization of concentration and density and particle size and thickness of layers.

Additional Embodiments:

6. The process as in claim 5 wherein said high absorption substance is deposited in a thin film containing high absorbing particles of density which assures that at least 60% of the light energy is intercepted and absorbed by the particles.

7. The process as in claim 5 wherein said high absorption substance is deposited in a thin film containing high absorbing particles of density which assures that at least 40% of the light energy is intercepted and absorbed by the particles.

8. The process as in claim 5 wherein said high absorption substance is deposited in a thin film containing high absorbing particles of density which assures that at least 20% of the light energy is intercepted and absorbed by the particles.

9. The process as in claim 1 wherein said high absorption substance is deposited in a thin film containing high absorbing particles of density corresponding to the rate of energy deposition per unit area so that the energy deposited in the skin is sufficient for the removal of at no more than 70% of the epidermis and the energy deposited in the skin allows permanent modification of the skin to a depth of no more then 100 micrometer below said depth of tissue removal.

Embodiment set #2 BIBI BIBI

1. A method for the removal of a superficial layer of a material while thermally modifying deeper layers, comprising the steps of:

Mixing a substance having a high absorption of at least one frequency band of electromagnetic radiation in a host substance, host substance has the properties that after mixing it can be formed into thin films or stripes, and,

Said stripes are capable of adhering to the surface of the target material thus becoming cohesive stripes, and

Applying said stripes to the target material,

Applying electromagnetic radiation to the area covered by the stripes with the substance having a high absorption of at least one frequency band of electromagnetic radiation being applied,

Embodiment set #3-BIBI BIBI

1. A method for applying energy to biological tissue, comprising: directing an electromagnetic energy source to apply the energy to a region of the tissue, so as to ablate a portion of the tissue in the region; and applying the energy to the region of the tissue comprises placing a material on the tissue to increase the absorption into the tissue of energy applied by the source.

2. The method of claim 1, wherein the material comprises: Water, glycerin, Stearic Acind, Aaloe Vera Gell, Isoparapffin Glycol Stearate, Mineral Oil, Lanolin, Cetyl acetate, Glycerl Stearate, Cetyl Alchohol, Dimenthicone, DEACetyl Phosphate, Magnesium Alluminium Sillicate, Acetyulated Lanolin Alcholol, Strearamide, AMP, Methylparaben, Propylpaaben, Fragrance, Cabomer 934, Disodium EDTA, Butylene Glycol, DMDM Hydantoin with a suspension of graphite powder.

3. The method of claim 2, wherein the graphite powder particles are larger than any pore in the human body

4. The method of claim 2, wherein the graphite powder particles are larger than about 30 micrometer

5. The method of claim 2, wherein the graphite powder particles are larger than about 60 micrometer

6. The method of claim 2, wherein the graphite powder particles are larger than about 1 micrometer

7. A method for applying energy to biological tissue, comprising: directing an electromagnetic energy source to apply the energy to a region of the tissue, so as to ablate a portion of the tissue in the region; and applying the energy to the region of the tissue comprises placing a material on the tissue to increase the absorption into the tissue of energy applied by the source.

8. The method of claim 7, wherein the absorption-enhancing material comprises graphite powder particles suspended in mineral oil.

9. The method of claim 2, wherein the absorption-enhancing material comprises graphite powder particles are suspended at a density of yyy particle per cm3

10. The method of claim 1, wherein the graphite powder particles are suspended in K-Y Jelly

11. The method of claim 1, wherein the material comprises: Water, glycerin, Stearic Acind, C11-13, Triethnolamine, Petrlatum, Sunflower See Oil, Soy Sterol, Lecithin, Sodium Stearoyl Lactylate, Tocopheryl Aceate (Vitamin E Acetate) Tetinayl Palmitate (Vitamin A Palmitate) Potassium Lactate, Urea, Colleagen Amino Acids, Sodium PCA, Lactic Acid, Zinc Oxide Isoparapffin Glycol Stearate, Mineral Oil, Lanolin, Cetyl acetate, Glycerl Stearate, Cetyl Alchohol, Dimenthicone, DEACetyl Phosphate, Magnesium Alluminium Sillicate, Acetyulated Lanolin Alcholol, Strearamide, AMP, Methylparaben, Propylpaaben, Fragrance, Cabomer 934, Disodium EDTA, Butylene Glycol, DMDM Hydantoin with a suspension of graphite powder

12. The method of claim 1, wherein the material comprises: Novocain and graphite powder suspension

13. The method of claim 1, wherein the material comprises: Antioxidant and graphite powder suspension.

14. A method for applying energy to biological tissue, comprising: directing an electromagnetic energy source to apply the energy to a region of the tissue, so as to ablate a portion of the tissue in the region; and applying the energy to the region of the tissue comprises placing a material on the tissue to increase the absorption into the tissue of energy applied by the source, said material is dispensed in such a concentration so as to allow approximately 100% of the incident energy to be converted to heat within the first interaction, followed by:

60% within the second interaction

30% within the third interaction, and

less then 5% within the forth interaction.

1. An apparatus for the ablation of a superficial layer of a tissue while thermally modifying deeper layers, comprising the steps of:

Mixing a substance having a high absorption of at least one frequency band of electromagnetic radiation in host substance, host substance has the properties that after mixing it can be formed into thin films or stripes,

Forming cohesive stripes of said host substance

Applying said stripes to the target tissue

Applying electromagnetic radiation to the area covered by the stripes with the substance having a high absorption of at least one frequency band of electromagnetic radiation being applied,

Additional Preferred embodiment Group A method for delivering an external substance through the outer skin barrier. BIBI BIBI

The method comprising,

Applying a substance to the surface of the skin,

Bringing an intermediate material in contact with the substance to be delivered

Applying an external energy to the intermediary material so that it is activated in a manner that causes said substance to be delivered to be able to move through the external surface of the skin into deeper layers.

Enhancement of Substance Delivery

A system for enhancing and improving the delivery of a substance across a barrier. A substance is applied to the surface of the barrier to be penetrated. The substance is then brought in contact with a device containing an energy source, said energy source emit energy which causes an intermediate medium to drive the substance across the barrier.

### PREFERED EMBODIMENTS

One set of preferred embodiment is:

1. A method for enhancing the transport of a substance through a barrier, comprising:

applying the substance to be transported to the surface of the barrier, and exposing the barrier to energy capable of driving said applied substance across the barrier.

2. The method of claim 1, wherein said energy capable of driving said substance across the barrier is also capable of modifying the barrier characteristics which enhance the penetration of said barrier by said substance.

3. The method of claim 1, wherein said energy capable of driving said substance across the barrier is also capable of modifying the barrier characteristics which enhance the penetration of said barrier by said substance, said energy is generated by an electromagnetic (EM) radiation source.

4. The method of claim 1, wherein said energy capable of driving said substance across the barrier is also capable of modifying the barrier characteristics which enhance the penetration of said barrier by said substance, said energy is generated by an electromagnetic radiation source and said electromagnetic radiation is subsequently absorbed by an intermediate material capable of absorbing said emitted EM energy.

5. The method of claim 1, wherein said energy capable of driving said substance across the barrier is also capable of modifying the barrier characteristics which enhance the penetration of said barrier by said substance, said energy is generated by an electromagnetic radiation source and said electromagnetic radiation is subsequently absorbed by an intermediate material capable of absorbing said emitted EM energy, said absorbed EM cause rapid expansion in at least a portion of said intermediate material, said rapid expansion drives said substance into and through the barrier, said rapid expansion also cause modification of the barrier properties, said modification enhance the delivery of said substance through the barrier.

6. The method of claim 1, wherein said energy capable of driving said substance across the barrier is also capable of modifying the barrier characteristics which enhance the penetration of said barrier by said substance, said energy is generated by an electromagnetic radiation source and said electromagnetic radiation is subsequently absorbed by an intermediate material capable of absorbing said emitted EM energy, said absorbed EM cause rapid expansion in at least a portion of said intermediate material, said rapid expansion drives said substance into and through the barrier, said rapid expansion also cause modification of the barrier properties, said modification enhance the delivery of said substance through the barrier, and,

said EM energy is focused from the source onto the intermediate substance and said focal spot is scanned across the intermediate substance surface.

7. The method of claim 1, wherein said energy capable of driving said substance across the barrier is also capable of modifying the barrier characteristics which enhance the penetration of said barrier by said substance, said energy is generated by an electromagnetic radiation source and said electromagnetic energy is subsequently absorbed by an intermediate material capable of absorbing said emitted EM energy, said absorbed EM cause rapid expansion in at least a portion of said intermediate material, said rapid expansion drives said substance into and through the barrier, said rapid expansion also cause modification of the barrier properties, said modification enhance the delivery of said substance through the barrier, and,

said EM energy is focused from the source onto the intermediate substance and said focal spot is scanned across the intermediate substance surface, and, scanning rate and scanning pattern determine the characteristics of the spatial and temporal modification of the barrier characteristics, and also determine the spatial and temporal driving effect on the substance being delivered across the barrier.

8. The method of claim 1, wherein said energy capable of driving said substance across the barrier is also capable of modifying the barrier characteristics which enhance the penetration of said barrier by said substance, said energy is generated by an electromagnetic energy source and said electromagnetic energy is subsequently absorbed by an intermediate material capable of absorbing said emitted EM energy, said absorbed EM cause rapid expansion in at least a portion of said intermediate material, said rapid expansion drives said substance into and through the barrier, said rapid expansion also cause modification of the barrier properties, said modification enhance the delivery of said substance through the barrier, and,

said EM energy is focused from the source onto the intermediate substance and said focal spot is scanned across the intermediate substance surface, and, scanning rate and scanning pattern determine the characteristics of the spatial and temporal modification of the barrier characteristics, and also determine the spatial and temporal driving effect on the substance being delivered across the barrier, and,

said intermediate medium is shaped so when it interacts with said EM energy, said interaction enhances the modification of the barrier to allow better delivery of said substance across the barrier, and said interaction also enhances the driving and delivery of substance across the barrier.

9. INDEP—A device for enhancing the transport of a substance across a barrier, the device comprising:

a dispenser for applying the substance to be transported to the barrier surface, an energy source, a conduit capable of directing energy from the energy source to said barrier's surface leading to enhancement of the delivery of said applied substance across said surface barrier.

10. The device of claim 1, wherein said energy capable of driving said substance across the barrier is also capable of modifying the barrier characteristics which enhance the penetration of said barrier by said substance.

11. The device of claim 1, wherein said energy enhancing the delivery of said substance across a barrier is also capable of modifying the barrier characteristics so that said modifications enhance the penetration of said barrier by said substance, wherein said energy is generated by an electromagnetic (EM) energy source.

12. The device of claim 1, wherein said energy enhancing the delivery of said substance across a barrier is also capable of modifying the barrier characteristics so that said modifications enhance the penetration of said barrier by said substance, wherein said energy is generated by an electromagnetic (EM) energy source, and wherein said electromagnetic energy is subsequently absorbed by an intermediate material capable of absorbing said emitted EM energy.

13. The device of claim 1, wherein said energy enhancing the delivery of said substance across a barrier is also capable of modifying the barrier characteristics so that said modifications enhance the penetration of said barrier by said substance, wherein said energy is generated by an electromagnetic (EM) energy source, and wherein said electromagnetic energy is subsequently absorbed by an intermediate material capable of absorbing said emitted EM energy, said absorbed EM energy causes rapid expansion in at least a portion of said intermediate material, said rapid expansion drives said substance into the barrier, said rapid expansion also cause modification of the barrier properties, wherein said effects of modification and driving of substance enhance the delivery of said substance through the barrier.

14. The device of claim 1, wherein said energy enhancing the delivery of said substance across a barrier is also capable of modifying the barrier characteristics so that said modifications enhance the penetration of said barrier by said substance, wherein said energy is generated by an electromagnetic (EM) energy source, and wherein said electromagnetic energy is subsequently absorbed by an intermediate material capable of absorbing said emitted EM energy, said absorbed EM energy causes rapid expansion in at least a portion of said intermediate material, said rapid expansion drives said substance into the barrier, said rapid expansion also cause modification of the barrier properties, wherein said effects of modification and driving of substance enhance the delivery of said substance through the barrier, and, wherein, said source EM energy is focused onto the intermediate substance while said focal spot is scanned across the intermediate substance surface.

15. The device of claim 1, wherein said energy enhancing the delivery of said substance across a barrier is also capable of modifying the barrier characteristics so that said modifications enhance the penetration of said barrier by said substance, wherein said energy is generated by an electromagnetic (EM) energy source, and wherein said electromagnetic energy is subsequently absorbed by an intermediate material capable of absorbing said emitted EM energy, said absorbed EM energy causes rapid expansion in at least a portion of said intermediate material, said rapid expansion drives said substance into the barrier, said rapid expansion also cause modification of the barrier properties, wherein said effects of modification and driving of substance enhance the delivery of said substance through the barrier, and, wherein, said source EM energy is focused onto the intermediate substance while said focal spot is scanned across the intermediate substance surface, wherein scanning rate and scanning pattern determine the characteristics of the spatial and temporal modification of the barrier characteristics, and also determine the spatial and temporal driving effect on the substance being delivered across the barrier.

16. The device of claim 1, wherein said energy enhancing the delivery of said substance across a barrier is also capable of modifying the barrier characteristics so that said modifications enhance the penetration of said barrier by said substance, wherein said energy is generated by an electromagnetic (EM) energy source, and wherein said electromagnetic energy is subsequently absorbed by an intermediate material capable of absorbing said emitted EM energy, said absorbed EM energy causes rapid expansion in at least a portion of said intermediate material, said rapid expansion drives said substance into the barrier, said rapid expansion also cause modification of the barrier properties, wherein said effects of modification and driving of substance enhance the delivery of said substance through the barrier, and, wherein, said source EM energy is focused onto the intermediate substance while said focal spot is scanned across the intermediate substance surface, wherein scanning rate and scanning pattern determine the characteristics of the spatial and temporal modification of the barrier characteristics, and also determine the spatial and temporal driving effect on the substance being delivered across the barrier, and, wherein,

said intermediate medium is shaped so when it interacts with said EM energy, said interaction enhances the modification of the barrier to allow better delivery of said substance across the barrier, and said interaction also enhances the driving of said substance across the barrier. 17. INDEP—A device for enhancing the transport of a substance across a barrier, the device comprising:

a dispenser for applying the substance to be transported to the barrier surface, a continuous wave electromagnetic energy source, a conduit capable of directing and scanning said electromagnetic energy from the EM energy source to said barrier's surface and moving it about said barrier surface thus leading to enhancement of the delivery and transport of said applied substance across said barrier.

18. The device of claim 17 wherein said energy from the EM energy source is focused onto an intermediate material comprised of at least two distinct surfaces, a first surface containing at least some high absorbing substance capable of absorbing at least some of said source energy and facing the energy source and,

a second surface made of peeks and troughs, said peaks form the “legs” which make contact with the surface of the barrier across which the substance is to be delivery, while the troughs contain the substance to be delivered and wherein the energy from the beam of EM energy source is scanned across the high absorbing substance surface generating rapid expansion in said high absorbing substance surface which drives the substance within each trough below said high absorbing surface into the barrier.

19. The device of claim 18 wherein said sources energy per unit area at the high absorbing substance is at least 10² W/CM² and a dwell time of on the order of 0.1 sec to 0.01 seconds.

20. The device of claim 18 wherein said sources energy per unit area at the high absorbing substance is at least 10⁴ W/CM² and a dwell time of on the order of 1 ms to 0.1 ms.

21. The device of claim 18 wherein said sources energy per unit area at the high absorbing substance is at least 10⁶ W/CM to 10⁸ W/CM² and a dwell time of on the order of 0.01 ms to 0.01 microsecond.

At power density of about 10⁴ W/cm², light interaction with matter begins to generate plasma and the interaction changes its characteristics. Characterization according to power density is therefore indicative of the type of interaction one may expect. Fluence level increase causes the intensity of the effect to change. For example, explosive ablation explosive events at a given plasma dominated regime will become more intense. [17]. Additionally, researcher used “thermal relaxation times” as a parameter for characterizing tissue effects according to a physical effect known as “thermal confinement”. The terms thermal relaxation time in this context really refer to the time it takes heat to travel outside of its optical deposition zone. This suggests that if heat is unable to diffuse out of the optical deposition zone, then the interaction is thermally confined. This was a source of some confusion since, of course, this assumes thermal energy removal by explosive/ablative events.

It is interesting to compare the characteristic Fluence (F) and Power densities (ρₚ) of two dominating lasers in cutaneous lasers surgery, the CO₂ and the Er:YAG. In the case of CO₂ operating at, for example, 40W and depositing its energy over an area 200 μm² within 1 ms ρₚ˜10⁵ W/cm² and a fluence of F˜10J/cm².

On the other hand, an Er:YAG laser with pulse duration of 250 μs and fluence of F˜10J/cm² would result in Power density of ρₚ˜4 10⁴W/cm².

The degree of thermal damage induced by these two lasers is still different because the water absorption of Er:YAG is about 10 times higher then that of the CO₂ and this normal mode laser in truth emits a train of short spiked μs pulses within the 250 μs envelop. However, from the above analysis based on power density, fluence and interaction time, one can see why thermal damage can be confined with both systems.

TSC utilizes laser sources that ranges in power from 1W to 40 W.

If a spot size of 1 mm×100 μm spot size is used, power densities, ρₚ, range from:

ρₚ=10³ W/cm² at 1W to 4 10³ W/cm² at 4W and to 4 10⁴ W/cm² at 40 W.

If a smaller spot size is used (for example 0.1 mm×0.1 mm) then all the above values are increased by a factor of 10 and range from:

10⁴W/cm² at 1W to 4 10⁵W/cm² at 40W

Fluence levels of the TSC depends on the dwell time and thus on the scan rates, and are given by:

F=P/(2νₓ1ₓy)

Where P is the source's power level, νₓ is the x-direction scanning frequency, 1ₓ is the length of the scan in the x-direction, and y- is the spot size in the y-direction.

For a 1W and 40W energy source and a spot size of 1 mm×0.1 mm, and with lₓ=1 cm and y=1 mm this corresponds to fluence levels outlined in Table 1:

TABLE 1

| | | | Fluence | Fluence |
|---|---|---|---|---|
| Scan | 1/2 | Dwell | J/cm² | J/cm² |
| Frequency | Period | time | (@ | (@ P = |
| (Hz) νₓ | (sec) | (ms) | P = 1 W) | 40 W) |
| 0.2 | 2.5 | 20 | 25 | 1000 |
| 0.4 | 1.25 | 10 | 12.5 | 500 |
| 1 | 0.50 | 4 | 5 | 200 |
| 2 | 0.25 | 2 | 2.5 | 100 |
| 4 | 0.125 | 1 | 125 | 50 |
| And @ | | The | 10³ | 410⁴ |
| Spot Size= | | power | W/cm² | W/cm² |
| 0.1*1.0 m | | density, | | |
| m² | | ρₚ is: | | |

The numbers given by the above analysis clearly demonstrate the tremendous versatility of TSC which can generate the same power densities, fluence level ranges of the Er:YAG and CO₂ with very similar tissue effects.

The analysis above provides two additional points of insight into applications of the TSC device. The BuffLight CLT applied to a skin surface of (6 mm)² and with average power of 40 W for a time duration of 100 ms, would provide the skin with a fluence of approximately 10 J/cm². Fluence ranging from 5J/cm² to 30 J/cm² (i.e. irradiation times of 50 ms to 300 ms, are effective for hair removal. In addition, a fluence of 10 J/cm² applied to a HAS penetrating the skin to a depth of 100 μm to 300 μm, will raise the temperature of that volume to by 250° C. to 80° C. Such high yet localized temperature increases have significant applications in the modulation of skin conditions as will be discussed later.

Since HAS can create a controlled optical energy deposition at the surface of the targeted skin, the subsequence interaction is highly controlled by the temporal and spatial condition created by the system. In the case of TSC, the amount of energy is highly controlled by several parameters:

1. The power output of the energy source.

2. The spatial extent of the energy distribution at the optical-to-thermal converting surface.

3. The energy distribution at the optical-to-thermal converting surface.

4. The time-dependence of the above energy distribution profile at the surface.

5. The time and space characteristics of the ERS.

TSC allows control over the above parameters by coordinating the power of the energy source, its off/on time, control of the beam spatial characteristics by manipulating optical components, and control over the ERS by synchronizing its action with the power source.

Controlling the above parameters allows the user of TSC to create a range of effects ranging from highly superficial surface cell removal substantially devoid of residual thermal damage (much like a typical 250 μs Er:YAG, or microdermabrasion systems) and all the way to very thermal CO₂-like tissue modification with depth of thermal damage raging several hundreds microns into the dermis. These versatile capabilities were clearly demonstrated by our histological studies.

BIBI BIBI MATTI MATTI YUV

### FIELD OF THE INVENTION

The present invention generally relates to a system for enhancing and improving the transcutaneous or transdermal delivery of various topical substances, chemicals or drugs.

BIBI BIBI BIBI BIBI

### FIELD OF THE INVENTION

The present invention relates generally to the application of energy to organic and non-organic materials, biological tissue, and more specifically to the application of energy to the skin.

### BACKGROUND OF THE INVENTION

It is known in the art to apply electromagnetic energy to biological tissue to engender changes therein. Sunbathers, for example, regularly expose themselves to bright sunlight in order to increase melanocyte activity in the basal layer of the epidermis, responsive to the sun's ultraviolet (UV) radiation. Artificial UV sources have been created to satisfy the desire for a “healthy”-looking tan in the winter. Other forms of electromagnetic energy, laser-light in particular, are currently used in a large range of therapeutic and cosmetic procedures, including eye surgery, hair removal, wrinkle removal, and tattoo removal.

PCT publication WO 98/55035, which is incorporated herein by reference, describes methods for minimizing injury to biological tissue surrounding a site exposed to pulses of electromagnetic energy.

U.S. Pat. No. 5,752,949 to Tankovich et al., which is incorporated herein by reference, describes a hair-removal method for placing a contaminant in the skin, using a laser to create explosions to drive the contaminant deep into hair ducts in the skin, and subsequently heating the contaminant to kill biological tissue surrounding the contaminant. The rate of application of energy is controlled to allow cooling of the skin.

U.S. Pat. No. 5,814,040 to Nelson et al., which is also incorporated herein by reference, describes a method for port wine stain removal, including applying a coolant to the skin, and subsequently directing laser radiation below the cooled area.

U.S. Pat. No. 5,810,801 to Anderson et al., which is also incorporated herein by reference, describes a method for treating wrinkles in the skin, including directing electromagnetic energy to a target region of the skin, while cooling an area above the target region.

### SUMMARY OF THE INVENTION

It is an object of some aspects of the present invention to provide improved apparatus and methods for applying energy to a material.

It is another object of some aspects of the present invention to provide improved apparatus and methods for removing heat generated during application of the energy to a material.

It is another object of some aspect of the present invention to apply the energy in a substantially controlled way and remove some of this energy in a controlled way in order to better control the damage to the material and to minimize pain when a human or animal tissue is the treated target material.

It is a further object of some aspects of the present invention to provide improved apparatus and methods for removing heat generated during application of electromagnetic energy to biological tissue.

It is still a further object of some aspects of the present invention to provide improved apparatus and methods for decreasing pain during application of electromagnetic energy to biological tissue.

It is yet a further object of some aspects of the present invention to provide improved apparatus and methods for performing medical treatments.

It is also an object of some aspects of the present invention to provide improved apparatus and methods for performing cosmetic treatments.

It is further an object of some aspects of the present invention to provide improved apparatus and methods for enabling a visible wavelength electromagnetic energy source to perform material and tissue removal and modification.

It is yet a further object of some aspects of the present invention to provide methods and apparatus for enabling a visible wavelength low-power electromagnetic energy source to perform material and tissue removal and modification.

It is still a further object of some aspects of the present invention to provide methods and apparatus for enabling a low-power electromagnetic energy source to perform material and tissue removal and modification.

It is also an object of some aspects of the present invention to provide improved methods and apparatus for enabling a low-power electromagnetic energy source to perform tissue removal and modification, substantially without pain, while controlling the amount of damage to remaining tissue.

In preferred embodiments of the present invention, an energy source applies electromagnetic energy to tissue of a subject, preferably so as to ablate a portion thereof. Thereafter, some of the heat generated by the interaction of the energy with the tissue is removed, typically by applying a coolant or a cooling element to the tissue. Removal of the heat immediately following the application of the energy generally reduces the subject's sensation of the heat, and, in particular, reduces any sensation of pain. Moreover, heat removal typically reduces or eliminates collateral injury to tissue surrounding the ablated area. Typically, although not necessarily, the tissue comprises the subject's skin.

It is known in the art to cool tissue prior to or during the application of electromagnetic energy to a subject's skin. U.S. Pat. No. 5,814,040 cited above, for example, describes cooling the epidermis prior to heating tissue thereunder, to minimize damage to the cooled tissue. Similarly, U.S. Pat. No. 5,810,801 describes cooling a first region of tissue while heating a second region of tissue thermally coupled thereto. These embodiments of the present invention, by contrast, teach that cooling the tissue following the application of electromagnetic energy is generally advantageous. Furthermore, it is believed that very rapid heating of a target area of the skin, substantially unmitigated by any prior cooling thereof, produces therapeutic results that are superior to techniques which cool a mass of tissue and then apply energy to a cooled target area. In particular, it is believed that the prior art methods that describe cooling of an upper tissue layer prior to interaction of electromagnetic energy therewith generally have an adverse effect on modification and/or ablation of tissue in the upper layer. Notably, unlike techniques as taught according to preferred embodiments of the present invention, these prior art methods generally are painful in the absence of an administered anesthetic.

In some preferred embodiments of the present invention, the energy source comprises a laser, whose beam is moved in a pattern over a target area of the skin. The pattern is made up of a sequence of passes over the tissue, each pass describing a line or curve over a predetermined portion of the target area. Typically, the pattern is chosen such that each pass is relatively distant from the preceding one, so that it generally does not cause additional heating of tissue indirectly heated by energy from the preceding pass. The temperature of this latter tissue decreases during the succeeding pass, preferably to approximately the same temperature as prior to the preceding pass. In some of these preferred embodiments, the laser energy is applied to the skin in sets of passes, whereby a set of one or more consecutive passes of the laser across the skin is relatively distant from a preceding set of one or more consecutive passes. The pattern is designed as necessary to ablate tissue, but minimize the undesired accumulation of heat in nearby tissue. Preferably, utilization of a beam pattern as described reduces sensations of pain, and also reduces injury of non-targeted tissue. In some of these embodiments, the tissue is actively cooled, preceding and/or following application of the energy thereto.

In some preferred embodiments of the present invention, a cover is placed over the target area, and the electromagnetic energy is applied through a window in the cover. Preferably, the cover enables an operator to maintain an ambient environment over the target area having properties, such as temperature, pressure, and humidity, which are set so as to minimize pain or discomfort during a procedure. Typically, sensors are coupled to the cover in order to determine parameters of the ambient environment and to generate signals responsive thereto. A control unit is preferably coupled to receive the signals and to actuate various devices so as to cause the signals to converge to desired values. The devices may include, for example, heating and cooling elements, pressure and humidity controllers, and a substance-delivery system. In a preferred embodiment, a temperature sensor monitors skin temperature, and active heating (e.g., using a laser) and cooling are applied as appropriate to keep the skin temperature within predetermined limits.

In a preferred embodiment of the present invention, the target area comprises two or more zones, typically concentrically arranged, and the electromagnetic energy source applies a different amount of energy to one of the zones from the amount applied to a second one of the zones. Preferably, the highest quantity of energy is applied to the innermost of the concentric zones, and successively smaller quantities of energy are applied to the other zones, responsive to their distance from the innermost zone. In this manner, a smooth transition is obtained from the target area to tissue or material surrounding the target area. By contrast, it is known in the art to use chemical or laser treatments for cosmetic purposes, such as wrinkle removal, but these treatments are typically disadvantageous, in that they often leave a region which clearly appears to have been treated, adjacent to a second region, which clearly has not been treated. For example, when these prior art methods are practiced to achieve wrinkle removal in the mustache area, a patch of generally wrinkle-free skin may be produced, but the patch is often sharply delineated from its neighboring, still-wrinkled skin.

In another preferred embodiment of the present invention, the target area comprises skin, and application of the energy thereto causes pores in the skin to expand. Pore expansion may be used to enhance transdermal drug delivery and/or as part of an acne treatment. Alternatively, drug delivery and acne treatment are performed responsive to the skin's ablation, irrespective of any pore expansion that may occur.

In some preferred embodiments of the present invention, a high absorption substance (HAS) is applied to the target area prior to activation of the electromagnetic energy source, so as to increase the absorption of energy in the target area.

There is therefore provided, in accordance with a preferred embodiment of the present invention, a method for applying energy to biological tissue, including: directing an electromagnetic energy source to apply the energy to a region of the tissue, so as to ablate a portion of the tissue in the region; and initiating cooling of tissue in the region subsequent to the ablation. In a preferred embodiment, initiating cooling includes thermoelectrically cooling.

In one preferred embodiment, directing the energy source to apply the energy includes generating a beam of energy having a diameter less than about 250 microns. Further preferably, directing the energy source to apply the energy includes generating a beam of energy having a dwell time over a point in the region of less than 25 ms.

In a preferred embodiment, initiating cooling includes applying a cooled surface to the tissue. Preferably, applying the cooled surface includes applying a cooled oscillating member to the tissue, which member is in contact with the tissue during a first phase of its oscillation and is not in contact with the tissue during a second phase of its oscillation.

Preferably, initiating cooling includes applying a coolant. Further preferably, the coolant includes a liquid and the method includes directing a flow of a gas towards a site on the tissue having the liquid applied thereto, so as to increase a rate of evaporation of the liquid.

In a preferred embodiment, the region includes a first region, and initiating cooling includes initiating cooling of tissue in the first region, wherein the method includes terminating cooling of the tissue in the first region subsequent to initiating the cooling thereof, and wherein the method includes directing the source to apply energy to a second region of the tissue so as to ablate a portion of the tissue in the second region subsequent to the initiation of cooling of the tissue in the first region. Alternatively or additionally, directing the source to apply the energy to the second region includes directing the source prior to the termination of the cooling of the tissue in the first region.

In a preferred embodiment, the region includes a first region, and ablating the portion includes ablating tissue to a first ablation depth in the first region. In this embodiment, the method includes directing the electromagnetic energy source to apply energy to a second region of the tissue adjacent to the first region, so as to ablate tissue in the second region to a second ablation depth, smaller than the first ablation depth. Typically, directing the source to apply energy to the second region includes smoothing the appearance of a border region between the first region and an untreated region of the tissue.

Preferably, the tissue includes skin, and the energy is applied so as to reduce a wrinkle in the skin and/or to decrease the size of a skin lesion. Alternatively or additionally, applying the energy includes expanding a pore of the skin, and, optionally, delivering a pharmaceutical product through the expanded pore.

In a preferred embodiment, the method includes applying a pharmaceutical product to the tissue. Typically, applying the product includes applying an anesthetic and/or an antibiotic.

Preferably, applying the energy to the region of the tissue includes placing a material on the tissue to increase the absorption into the tissue of energy applied by the source. Further preferably, the material includes a substance characterized by high absorbency of energy of a wavelength generated by the source. Still further preferably, the energy source includes a CO2 laser. Alternatively or additionally, the output of the energy source is less than about 5 W.

In a preferred embodiment, the electromagnetic energy source includes a broadband emission lamp. Alternatively or additionally, the electromagnetic energy source includes a laser, typically a CO2 laser, an Er:YAG laser, a microchip laser, and/or a diode laser. Preferably, the diode laser has a power output of less than about 500 mW.

Preferably, the method includes actively warming cooled tissue responsive to the cooling, so as to decrease injury of the cooled tissue. Typically, warming includes sensing a temperature of the tissue and warming the tissue responsive thereto. In a preferred embodiment, warming includes applying a heated gas to the tissue. Alternatively or additionally, warming the tissue includes thermoelectrically warming the tissue, and initiating cooling includes thermoelectrically cooling the tissue. Further alternatively or additionally, warming includes applying a heated surface to the tissue. In a preferred embodiment, warming the tissue includes directing the energy source to apply additional energy to the cooled tissue, which additional energy substantially does not cause ablation. Typically, applying the additional energy includes enlarging a beam diameter of the energy source from a first diameter, used for ablating tissue, to a second diameter, used for warming tissue.

There is further provided, in accordance with a preferred embodiment of the present invention, a method for applying energy to biological tissue, including: directing an electromagnetic energy source to apply energy along a first path on the tissue, so as to ablate tissue in the path;

directing the source to apply energy along a second path on the tissue, relatively distant from the first path, so as to ablate tissue in the second path, while allowing cooling of tissue adjacent to the first path heated by diffusion due to applying the energy to the first path; and

directing the source to apply energy along a third path on the tissue, closer to the first path than to the second path, so as to ablate tissue therein.

Preferably, applying the energy along the first path includes placing a material on the tissue to increase the absorption into the tissue of energy applied by the source. Further preferably, the material includes a substance characterized by high absorbency of energy of a wavelength generated by the source.

In a preferred embodiment, the method includes actively cooling tissue in a vicinity of the first path. Preferably, actively cooling includes initiating the active cooling subsequent to ablation of the tissue in the first path. Alternatively or additionally, actively cooling includes applying a coolant in the vicinity of the first path. Further alternatively or additionally, actively cooling includes thermoelectrically cooling. Still further alternatively or additionally, actively cooling includes applying a cooled surface to the tissue in the vicinity of the first path. Typically, applying the cooled surface includes applying a cooled oscillating member to the tissue in the vicinity of the first path, which member is substantially not in contact with the tissue in the vicinity of the first path during a first phase of its oscillation and is in contact with the tissue in the vicinity of the first path during a second phase of its oscillation. Directing the electromagnetic energy source to apply energy along the first path on the tissue generally includes directing the source during the first phase.

In a preferred embodiment, the method includes applying a pharmaceutical product in a vicinity of the first path.

There is still further provided, in accordance with a preferred embodiment of the present invention, a method for applying energy to biological tissue, including:

placing a cover on the tissue, and

directing a beam of electromagnetic energy through the cover to impinge on the tissue, so as to ablate a portion thereof.

Preferably, the cover includes a window, through which the energy beam passes.

Further preferably, placing the cover on the tissue reduces stimulation of nerves therein. Typically, placement of the cover defines a volume of an intermediate substance, between the cover and the tissue, and reducing stimulation includes maintaining a desired characteristic of the intermediate substance. The substance may include air and/or a liquid. In a preferred embodiment, the characteristic includes a temperature, a pressure, and/or humidity.

There is yet further provided, in accordance with a preferred embodiment of the present invention, a method for applying energy to biological tissue of a subject, including:

directing an electromagnetic energy source to apply the energy to a region of the tissue, so as to ablate a portion of the tissue in the region;

applying a liquid coolant to the tissue; and

directing a flow of a gas towards the tissue, so as to increase a rate of evaporation of the liquid coolant.

Typically, the gas is directed to the tissue so as to reduce a perception of discomfort by the subject responsive to the coolant.

There is also provided, in accordance with a preferred embodiment of the present invention, apparatus for applying energy to biological tissue, including:

an electromagnetic energy source, directed to apply the energy to a region of the tissue, so as to ablate a portion of the tissue in the region; and

a cooling unit, which initiates cooling of tissue in the region subsequent to the ablation.

In a preferred embodiment, the cooling unit includes a thermoelectric cooling unit.

Alternatively or additionally, the apparatus includes a pharmaceutical delivery unit, containing a pharmaceutical product for application to the tissue in a vicinity of the ablated portion.

Further alternatively or additionally, the apparatus includes a heating unit, which heats tissue cooled by the cooling unit, so as to decrease injury of the cooled tissue. Preferably, the apparatus includes a temperature sensor, which generates a signal responsive to a temperature of the tissue, wherein the heating unit heats the tissue responsive to the signal. In a preferred embodiment, the heating unit includes a thermoelectric element, through which element current is driven in a first direction so as to heat the tissue. Preferably, the cooling unit drives current through the thermoelectric element in a second direction so as to cool the tissue. Alternatively or additionally, the heating unit includes a heated surface which is applied to the tissue.

There is additionally provided, in accordance with a preferred embodiment of the present invention, apparatus for applying energy to biological tissue, including:

an electromagnetic energy source, which applies energy to the tissue; and

a beam scanner, which directs the energy from the source along a plurality of paths on the tissue, including (a) a first path, so as to ablate tissue in the first path, (b) a second path, relatively distant from the first path, so as to ablate tissue in the second path, while allowing cooling of tissue adjacent to the first path heated by diffusion due to applying the energy to the first path, and (c) a third path, closer to the first path than to the second path, so as to ablate tissue in the third path.

There is yet additionally provided, in accordance with a preferred embodiment of the present invention, apparatus for applying energy to biological tissue, including:

a cover, placed on the tissue; and

an electromagnetic energy source, which directs a beam of energy through the cover to impinge on the tissue, so as to ablate a portion thereof.

Preferably, the apparatus includes a pharmaceutical reservoir, containing a pharmaceutical product for application to the tissue.

There is also provided, in accordance with a preferred embodiment of the present invention, apparatus for applying energy to biological tissue of a subject, including:

an electromagnetic energy source, directed to apply the energy to a region of the tissue, so as to ablate a portion of the tissue in the region; and

a cooling unit, which applies a liquid coolant to the tissue and directs a flow of a gas towards the tissue, so as to increase a rate of evaporation of the liquid coolant.

Preferably, the cooling unit directs the gas to the tissue so as to reduce a perception of discomfort by the subject responsive to the coolant.

There is additionally provided, in accordance with a preferred embodiment of the present invention, apparatus for applying energy to biological tissue, including:

An energy source,

A substance capable of absorbing said energy source

Said substance also capable of conducting the absorbed energy to a target material

An element capable of REMOVING the energy from said absorbing material

The present invention will be more fully understood from the following detailed description of the preferred embodiments thereof, taken together with the drawings, in which:

### BRIEF DESCRIPTION OF THE DRAWINGS

FIG. 1.1 is a simplified pictorial illustration showing an apparatus for modifying a target material, in accordance with a preferred embodiment of the present invention.

FIG. 1.2 is a graphic illustration of an exemplary intermediary energy absorbing material that can be mounted on two revolving mounts designed to advance the intermediate material following each use.

FIG. 1.2.1 is a graphic illustration of an exemplary intermediary energy absorbing material that can be mounted on two revolving mounts designed to advance the intermediate material following each use. The photograph also illustrates the handpiece containing the energy source and the energy-removing component. The cylindrical bottle contains the energy removing substance or coolant.

FIG. 1.2.2 is a photograph illustrating an exemplary intermediary energy absorbing material that can be mounted on two revolving mounts designed to advance the intermediate material following each use. The photograph also illustrates the handpiece containing the energy source and the energy-removing component. The cylindrical bottle contains the energy removing substance 148 or coolant. The red dot on the intermediate material is the beam of energy being absorbed by the intermediate material. The larger consol 149 contains the electronics and control allowing synchronization of the energy source and energy removal element.

FIG. 1.3 shows a heater embedded in the intermediate material. The Energy removal source is either acting through the intermediate medium or is external to the intermediate medium (IM) and effect energy removal. The energy removal can also act directly on the target material.

FIG. 1.4 shows a heater embedded in the intermediate material. The Energy removal source is also embedded in the intermediate material and consist of a coolant coming from a coolant reservoir and circulating in a tube in contact with the intermediate material, thus capable of cooling the intermediate material.

FIG. 1.5 by creating cuts in the intermediate material and creating an alternating series of HCM isolated, one can create a series of selected burn. Alternatively, one can have the ENTIRE intermediate material made of HCM, while only a certain region covered with HAS. This will ensure selective heating while uniform cooling.

FIG. 1.6 intermediary energy absorbing material can be mounted on a revolving disk.

FIG. 1.7 is a heater cooling combination where the cooling cools both the target tissue and the energy source during operation of the energy source.

FIG. 1A is a simplified pictorial illustration of an apparatus for modifying and removal material and biological tissue, in accordance with a preferred embodiment of the present invention.

FIG. 1B is a simplified pictorial illustration of an exemplary pattern for depositing High Absorption Substance (HAS) on the intermediate material of the apparatus for modifying material and removal of biological tissue, in accordance with a preferred embodiment of the present invention.

FIG. 1C illustrates the pattern and direction of heat diffusion following the energy deposition in the pattern of HAS of FIG. 1B.

FIG. 1D illustrates an exemplary pattern for High absorbing substance placed on the intermediate material.

FIG. 1E illustrates the extent of thermal diffusion of the energy in the target material in response to the energy deposition in the high absorbing substance pattern in the intermediate material as shown in FIG. 1B.

FIG. 1F illustrates an exemplary pattern for High absorbing substance placed on the intermediate material and the corresponding thermal energy deposition in the target material.

FIG. 2A is a simplified pictorial illustration showing an apparatus for modifying a target material, in accordance with a preferred embodiment of the present invention.

FIG. 2B is a simplified pictorial illustration showing an apparatus for modifying a target material, allowing the intermediate material to be lifted to allow energy removal directly from the target material.

FIG. 3A is a simplified pictorial illustration showing an apparatus for modifying a target material.

FIG. 3B is a simplified pictorial illustration of possible patterns and shapes of the high energy absorbing substance.

FIG. 3C is a simplified pictorial illustration showing additional possible patterns and shapes of the high energy absorbing substance.

FIG. 4 is a simplified pictorial illustration showing an exemplary device for material removal and modification and the device related controls.

FIG. 5 is a simplified pictorial illustration showing in greater details an exemplary control box for the device for material removal and modification.

FIG. 6 is a simplified pictorial illustration showing in greater details an exemplary device for material removal and modification.

FIG. 7A is a simplified pictorial illustration showing an exemplary method for material removal and modification using a stationary beam method.

FIG. 7B is a simplified pictorial illustration showing an exemplary method for material removal and modification using a moving beam method.

FIG. 7C is a simplified pictorial illustration showing an exemplary method for material removal and modification using a broad beam method.

FIG. 7D is a photograph showing an exemplary cap with an exemplary intermediate material used in a device for material removal and modification.

FIG. 7E is a photograph showing an exemplary cap with an exemplary intermediate material mounted on a box containing the energy source and energy removal element used in a device for material removal and modification.

FIG. 7F is a simplified pictorial illustration showing a time-sequence of energy deposition, energy removal and additional energy deposition as practiced by a preferred embodiment of the present invention.

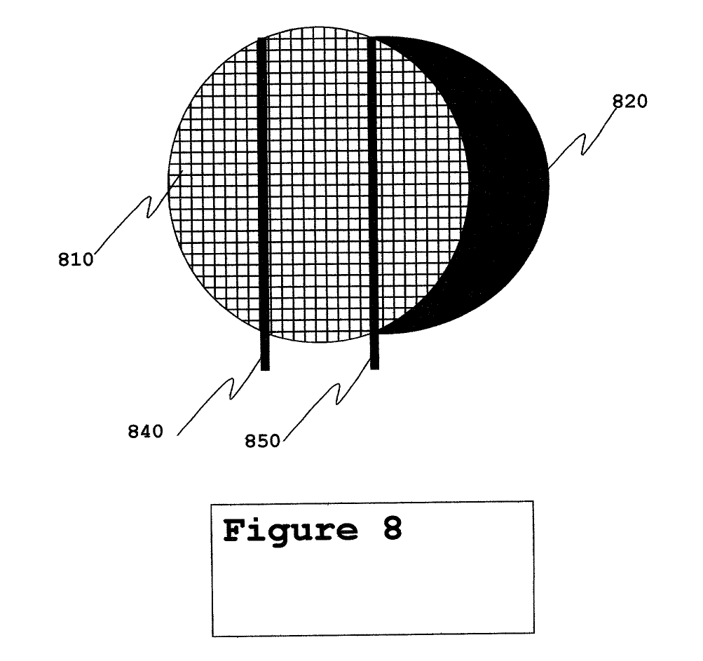

FIG. 8 is a simplified pictorial illustration showing the method of peeling intermediate material used in the present invention for target material removal and modification.

### DETAILED DESCRIPTION OF PREFERRED EMBODIMENTS

FIG. 1.1 is a simplified pictorial illustration showing an apparatus 22 for modifying a target material, in accordance with a preferred embodiment of the present invention. Apparatus 22 comprises an energy source 32, which delivers energy to a target material 62, so as to remove or modify a layer of the target material. The apparatus consist of an energy source 32, an energy coupling element 52 coupling the source of energy to the target material, and an energy-removing element 72. Preferably, the energy-removing element is coupled to the energy source so that the energy deposition and energy removal are synchronized.

Alternatively, in another preferred embodiment, an intermediate material made of thermally conducting material (for example, Aluminum) is patterned to so that heating occurs only in one area. That heat travels very fast to all other locations and so that heat travels along conduits to specific areas for heating and modification of the target material and then cooling quickly removes the heat from the target. The high absorbing substance on an intermediate conducting material can indicate how deep the thermal effect in the target material has reached

In another preferred embodiment, the energy source beam can rotated along a circle made of spots of high absorbing substance. Much like a drum gun the beam is shifted each time it fires. This we can allow the user, a precise, predetermined number of uses and no more. Once the high absorbing substance on the spots in the circle is consumed no further interaction can occur. This is illustrated in FIG. 1A.

In another preferred embodiment, the energy intermediary absorbing material can be mounted on two counter-revolving mounts as shown in FIG. 1.2, which can be adjusted (either manually or automatically) to replace the high absorbing substance as it is being consumed by the interaction. Here the attachment 116, attached to the output port in the handpiece, consists of two rotating mounts 126, which are both rotating in the same sense in order to advance a film of high absorbing substance 156. The High absorbing substance is deposited in “frames” 136 which are deposited on the film band 156 which is then advanced frame by frame to place a fresh frame before each new area treatment. The spot 146 represent an exemplary energy beam from a light source interacting with the high absorbance substance.

FIG. 1.1 is a simplified pictorial illustration showing an apparatus 22 for modifying a target material, in accordance with a preferred embodiment of the present invention. Apparatus 22 comprises an energy source 32, which delivers energy to a target material 62, so as to remove or modify a layer of the target material. The apparatus consist of an energy source 32, an energy coupling element 52 coupling the source of energy to the target material, and an energy-removing element 72. Preferably, the energy-removing element is coupled to the energy source so that the energy deposition and energy removal are synchronized.

Alternatively, in another preferred embodiment, an intermediate material made of thermally conducting material (for example, Aluminum) is patterned so that heating occurs only in one area. The heat travels rapidly to all other designated locations along the manufacturer-designed thermal conduits to specific areas for heating and modification of the target material. Controlled cooling is then activated and quickly removes the heat from the target. A high absorbing substance on an intermediate conducting material can control how deep the thermal effect in the target material has reached

In another preferred embodiment, the energy source beam can be rotated along a circle made of spots of high absorbing substance. Much like a drum-gun, the beam is shifted each time it fires. This we can allow the user, a precise, predetermined number of uses and no more. Once the high absorbing substance on the spots in the circle is consumed no further interaction can occur. This is illustrated in FIG. 1A.

In another preferred embodiment, the intermediary energy absorbing material can be mounted on two counter-revolving mounts as shown in FIG. 1.2, which can be adjusted (either manually or automatically) to replace the high absorbing substance as it is being consumed by the interaction. Here the attachment 116, attached to the output port in the handpiece, consists of two rotating mounts 126, which are both rotating in the same sense in order to advance a film of high absorbing substance 156. The High absorbing substance is deposited in “frames” 136 which are deposited on the film band 156 which is then advanced frame by frame to place a fresh frame before each new area treatment. The spot 146 represent an exemplary energy beam from a light source interacting with the high absorbance substance.

BIBI BIBI BIBI

In an alternative preferred embodiment the intermediate medium is replaced at the required interval by rotating a carousel, or a rotating wheel at the required interval to a new “fresh” HAS-coated, location. This is shown in FIG. 1.6. Here, the apparatus is identical to the attachment FIG. 1.2 except that instead of the rollers one use a wheel 226, where the high absorbing substance is deposited on the surface of the wheel facing the energy beam, 146 as shown in FIG. 1.6. The wheel is then rotated before each interaction to allow new and un-used high absorbing substance surface to be placed between the energy beam 146 and the target material

In yet another exemplary preferred embodiment, the energy can be delivered to the target material 1160, in an energy source 1130 embedded in the intermediate material 1140. FIG. 1.3 shows a heater 1130 embedded in the intermediate material 1140. The Energy removal element 1150 is then either embedded in the intermediate medium as shown in FIG. 1.4, or is external to the intermediate medium (as shown in FIG. 1.3) and allows energy removal. Energy removal can then be generated either through the intermediate material as shown in FIG. 1.4 or by direct application of energy removal substance to the intermediate medium as is shown in FIG. 1.4. In both cases the reservoir 1155 containing the energy removal substance is also shown. Alternatively, in yet another preferred embodiment, the energy removal substance may also be applied to directly to the target material 1160 thus removing energy directly from the target material.

The use of a highly controlled heating and cooling is contemplated. For example, one preferred embodiments is illustrated in FIG. 1A: A thin, highly conducing intermediate material (HCM, for example a thin sheet of Aluminum) 20 is attached to a target material such as the skin 10. The HCM is coated at specific points with high absorbing substance 30 (HAS). The laser beam is directed towards one such location of high absorbing substance-coated material. The heat is absorbed by the HAS and conducted through the layer of highly conducting material. For example we calculate that in a circular area of 1 cm² heat will be diffuse to a depth of about 600 um in about 0.36 sec or 360 ms, in water-like substance. The energy source 40 is on for the duration of time corresponding to power of the energy source allowing sufficient energy to be deposited. Immediately following the interaction a coolant spray 60 from a coolant ejector 50 is discharged allowing immediate cooling of the target area through the HCM film. One can design the film thickness and size so that the resultant energy per unit time is sufficient to achieve the desired effect.

In 360 ms the thermal diffusion in a water-like tissue target material is on the order of 600 um. On the other hand a circular area of 3 mm² will be evenly heated up by a scanned energy source contemplated in one preferred embodiments of this invention, in about 36 ms by an exemplary scanning of the optical light. During this time, heat diffuses only a ˜200 um. This will be more in line with the thermal tissue damage that we wish to take place. Such a small volume, small size intermediate material will allow the use of a single stationary beam. One can then use a MECHANICAL device (for example a drum-like switcher (much like in a revolver pistol) that will move small, mirrored faces 47 to move the beam 48 around to the various spots of high absorbing substance 30.

Alternatively the intermediate material 20 in FIG. 1A can be made of a THERMALLY NON-CONDUCTING thin intermediate medium. The highly absorbing substance 30 is deposited on the intermediate material and the process continues as described above. The main difference between the two methods is that very little lateral conduction occurs with the intermediate material 20 made of Non-conducting material. On the other hand, efficient transfer of thermal energy from the HAS spots 30 to the target material is ensured by using a very thin layer of 20. This has the advantage that while no photons or high absorbing substance comes in direct contact with the target material, the heat is quickly and efficiently transferred to the target and can then be just as efficiently and quickly eliminated.

In either one of the above preferred embodiments, the intermediate substance can be made so there is no direct contact or interaction of the energy from the energy source (for example, photons from an electromagnetic energy source) NOR direct contact of the HAS with the material. For example, only the sterile outer surface of the intermediate material makes direct contact or has direct interaction with the target material.

In the embodiment that envisions a non-conducting intermediate material, the highly absorbing substance, HAS, can be precisely deposited at the selected locations on top of the intermediate material. Since thermal diffusion in the lateral direction is very limited the interaction is confined to substantially the area where the HAS is exposed to the energy beam. For example, if the non thermally-conducting intermediate material is a water-like material with water-like thermal properties, with approximately 100 um thickness, it will conduct thermal energy through in about 10 ms. The thermal interaction with the target material will start within less then 10 ms and the lateral heat diffusion, if, for example, we deploy the energy removal system within 40 ms, will be confined to boundary of 200 um from the energy deposition zone (i.e. the lateral distance from the zone where the HAS is deposited).

In fact, by controlling the lateral spatial separation between the HAS zones one can create a continuum of thermal deposition. This is shown in FIG. 1F where the pattern 82 of highly absorbing substance 30 deposited on the intermediate medium 20, becomes after thermal energy diffusion, a substantially uniform target-material modification pattern, 85, on the target material 10.

Similarly the process can contain sources other then light (for example a broad band light source, a xenon or halogen lamp) in synchronized use with the coolant.

In one such preferred embodiment, a thermo-electric cooler (TEC) can be employed. Here the TEC is off during the energy deposition phase. The TEC is then turned on to remove energy and quench the interaction at a precisely desired moment. Alternatively, in another preferred embodiment, the TEC can also act as an energy source and heat up the targets material, then the electric current polarity is reverse and the TEC can then acts as an energy removal element and rapidly cool the target material.

However, cryogen spray is more easily and rapidly controlled. In yet another preferred embodiment, the TEC polarity may be reversed rapidly and in a controlled manner upon the generation of a signal from the user or a controller unit. In this embodiment, the TEC acts as both the energy source and as an intermediate material. Heating of the target material takes place and modification of the target material follows. The TEC controller then causes heating of the target material to stop at a desired moment in time, and the removal of energy and/or cooling to follow in order to quench the interaction.

In yet another preferred embodiment a thermoelectric cooler can be combined with the process of coblation or electric-cautery or electric-surgery to achieve rapid energy removal following energy deposition in order to minimize pain and control damage and control target material modification. The same process can also be applied to the process of coblation where energy removal and minimization of pain can be achieved by application of energy removing to the target material after any and all plasma mediated interactions. In this embodiment, the energy removing can be achieved by using thermo-electric cooler, a peltier cooler, coolant spray, cryogen spray as well as a host of other energy removal mechanism. All of these will work provided that the user allow the modifying interaction to take place and then, at a predetermined point in time, the user activate one of the above energy removal methods. This applies to the processes of ablation that can be envisioned as well except that ablation takes place by electro-surgery or Coblation or plasma generation, or plasma-mediated material removal, or rapid generation of heat. Again, cooling occurs either THROUGH the highly conducting material (for example if aluminum is used), or if electrodes are used for delivering electric, electromagnetic, irradiative, or chemical energy. And it will also work if one raises the intermediate material to allow directed application of the energy removing substance (or coolant) to the target material as shown in FIG. 2B where the intermediate material 340 is lifted up to allow the energy removal substance to be applied directly to the targeted tissue 10 following the interaction. This also ensures minimization of pain with all methods including electro-cautery/hyfercator.

In yet another preferred embodiment, the intermediate medium consists of an electrical heater in contact with the target material. The electrical heater may be pulsed or may be on continuously. IF it is on continuously, a pulsed energy removal system is employed. For example, a sufficiently think intermediate material, said intermediate material is also a heater or capable of heating the target material, which is then sprayed by a cryogen spray or cooled by other coolant or energy removal means in order to modulate both intermediate material AND target material temperature and thermal energy content. The cryogen spray will also remove thermal energy from the target material through the intermediate medium. This will allow reduction of the target material temperature at the desired times.

With the above methods we can also aid in the removal of pigmented skin blemishes that can also be affected. This can be done much like the CO2 only much less expensive, pain-free, AND with a much better control of spatial targeting because of the high absorption substance deposition.

Another advantage of the embodiment envisioning the depositing of high absorbing substance (HAS) on a paper or thermally mediating medium but on the side-away from the skin is that the intermediate material or intermediate paper block any debris from the ablated target material. In addition, the high absorbing substance is not allowed to come in contact with the skin.

Attached below is an exemplary list of possible intermediate papers and or material that can serve as intermediate material:

A.

The intermediate material can be prepared with the high absorbing substance embedded in it.

Colored paper

Toilette paper

Tissue paper

Paper matrix with the HAS embedded in its matrix.

B.

Good conductors which may for example be shaped into a foil

Stainless steel

Steel

Aluminum,

Copper

Gold

Other metals

The above method means containing both light and HAS within the Device. The target material or skin is never in direct contact with the material and is never exposed the photons from an energy source.

C.

An intermediate material for the delivery of energy may be devices such as hyfercator or electrocauterers with Cooling apparatus for energy removal

D.

Coblation with Cooling

E.

An alternative method may be a thermoelectric cooler with a laser acting as an energy source. The laser radiation may be activated in a pulsating mode while the energy removal (e.g. cooling) is synchronized with the heat source.

F.

Energy may be pumped into the intermediate material using high resistance electrical wires. The intermediate material can then be cooled rapidly with a loop of thermally conducting tube containing a circulating cryogen or other water flow, or through the use of cryogen spray.

Another embodiment is shown in FIG. 2A—The Coolwand™

The energy source (for example a laser source) 300 and the coolant source 310 are mounted inside the handpiece 320. The cap 330 is made of a conducting material or from a material thin enough to allow significant heat conduction from the inside part of the cap 330 to the outside part 335 in a short time—substantially shorter then about 0.5 second. The power supply 340 powers the instrument's energy source and cooling source.

The light from the energy source, (e.g. a laser or a broadband light source) is absorbed by a high absorbing substance (HAS) 350. The HAS 350 transfers its energy to the conducting part 355 which stretches throughout the intermediate material cap volume 370 is in contact with the target material 10. The rest of the intermediate material cap volume 370 is made of insulating material. Thus, by controlling the size and volume of the high conductive substance area=the high thermal conductivity area size 340, one determines how the size of the area to be affected. By controlling the power and the on time duration of the energy source, one determines how much energy will be delivered to what size area. By controlling the coolant, one determines the duration of time for which the interaction is allowed to go on. Thus, this embodiment allow for elimination of scanners, no moving parts, no contact of the HAS with the skin, and no exposure of the skin to photons or any energy other then highly controlled thermal energy. There is also no microprocessors or programs to control this. It can all be made with hardwired design.

The high absorbing substance 350 is consumed within a given amount of time and thus allows a finite amount of time for use. The Size of the high conductive material (HCM) 355 is adjustable to treat different areas shape and sizes. Each HCM 355 is packed within its own cap 330 making it a disposable cap. The Energy source power and duration are presets and corresponds to the size of the HCM size and design.

FIG. 1A illustrates one of the preferred embodiments:

A source of energy 40

Deliver a dose of energy to an intermediate material 20.

The intermediate substance absorbs this energy and conducts it to the target material 10.

It is also possible that the intermediate substance 20 may have on it a few absorbing locations 30 where a substance of high absorbance with respect to the energy of the source 40 has been applied. This energy is then absorbed by the high absorbing substance locations 30 which then transfer them to the intermediate material 30 which then transfer the energy to the target material 10. There, said energy modifies or ablate the target material 10. Subsequent to the interaction, (or, in some preferred embodiments during) a substance capable of energy removal 70 is directed by the ejector/director 50 to the intermediate material 20 which is in contact with the target material 10. The substance capable of energy removal so directed 60, then remove at least some of the energy from the intermediate material 20 and thus also from the target material. This procedure allows, among other things minimization of pain in human tissue interaction and greater control of damage.

In further elaboration of this embodiment, the high absorption substance (HAS) 30 may be located at several different points thus allowing efficient delivery of energy to various locals on the intermediate material and target material. This too is illustrated in FIG. 1A.

In a further elaboration of this embodiment, the high absorption substance (HAS) 30 may be located at several different points 30 thus allowing efficient delivery of energy to various locals on the intermediate material and target material. This will also spread out the energy deposition effect and will allow simultaneous deposition of energy in one location AND removal of energy in a second, adjacent location. Of course the energy source 40, in this embodiment has to be stirred from one location 30 to the next in a predetermined pattern. The stirring of the energy source can be accomplished by means of an energy-stirring device 45. The energy removal substance ejector/director 50 can be synchronized with the beam stirring device 45 to accomplished the above mentioned simultaneous energy deposition in one location and energy removal in a second location.

In a further embodiment the intermediate material 20 may be made of a conductor or an insulator. If a conductor—then depending on the thickness, of the plate, it may conduct well within a given interaction time (i.e before the energy removing substance ERS is applied) to all region of the plate 20. However if the plate is very thin, the flow of energy is limited by the cross section and is limited in the lateral direction. Energy flow will be efficient in the Z-direction (i.e. towards the target material). I.e., most of the energy shall be conducted directly, in the Z-direction towards the target material and very little energy shall be conducted laterally and then into the targeted material. The energy distribution in the intermediate material will be that shown by 65 in FIG. 1B. Then, due to the finite thermal conduction in the intermediate material the final thermal energy distribution in the target material, will be that shown by 70 in FIG. 1C where the concentric arrows indicate the direction diminishing thermal effect.

In another preferred embodiment, the intermediate material is made of a substantially thin thermal insulator. The energy from the energy source is then applied to the intermediate material via a high absorbing substance 30 applied at any desired pattern as shown in FIG. 1D. Since the absorbed source energy is now converted to thermal energy but remains confined substantially to the shape of the HAS spots 30 on the intermediate material 20, at least some of this energy will be transferred to the target material 10 below, shown in FIG. 1E and diffuse further down towards the target material. If said target material is composed, for example, of tissue or water-like material, the thermal diffusion will be rather slow (for example it takes about 10 ms in such water-like material for thermal energy to diffuse a distance 100 um) and the thermal effect at the target by the time the energy removal system (or coolant) is activated, will be more confined, for example, to within the second ring, 75, in FIG. 1E.

An additional preferred embodiment envisions the energy source as a rapid heater embedded within confining caps. These heaters may be made of electrical heaters. Alternatively, these heaters may be made of thermoelectric cooler with switchable polarity.

An alternative embodiment is shown in FIG. 2A: The energy source (for example, a laser) 300 and the energy removal source (for example a coolant) 310 are mounted inside a handpiece 320. The intermediate material 330 is shaped like a cap and is made of a conducting material or from a material thin enough to allow sufficient amount of heat to be conducted from the inside surface of the cap 330 to the outside surface of the cap in contact with the target material 335. Preferably this conduction is completed in a short time for example shorter then about 0.5 second. The power supply 340 powers the instrument's energy source and cooling source.

The light from the energy source (e.g. a laser or a broadband light source) is absorbed by a high absorbing substance 350. The high absorbing substance 350 transfers its energy to the conducting part 340 which is in contact with the skin 360. The rest of the cap area 370 is made of an insulating material, which does not conduct thermal energy well. Thus, by controlling the size of the material of high thermal conductivity (HCS) 340, one can determine how the size of the area to be affected. By controlling the power and the “on” time duration of the energy source, one determines how much energy will be delivered to a desired area size. By controlling the coolant duration and timing, one determines the duration of time for which the interaction is allowed to go on.

There are no scanners, no moving parts, no contact of the high absorbing substance with the skin, and no exposure of the skin to photons or any energy other then highly controlled thermal energy. There are also no microprocessors or programs needed to control the process. All operations are hardwired.

The highly absorbing substance 350 in FIG. 2A, is consumed within a given amount of time and thus allows only a well-defined finite length of time for use.

The Size of the highly conducting material 355 is adjustable to treat different areas, shapes, and sizes. Each highly conducting material 355 is packed within its own cap 370, and since the highly absorbing material is at least partly consumed by during operation, the caps are disposable caps.

The Energy source power and duration settings are presets and are designed to correspond to the size and shape of the highly conductive material.

FIG. 3A illustrates a replaceable cap that can be attached to an exemplary device for modification of target material, 3030. The caps 3035, constitute the intermediate material that absorbs the source's radiation and conduct it to the targeted surface. The cups may be arranged so that a certain portion of them is highly conductive and a certain portion of them is highly insulating. Further, part of the conductive portion of the cup may be coated with capable of absorbing well the energy from the source. For example in FIG. 3A, the energy from the source is scanned across the cup's surface 3040 that is facing the energy source 300. The regions that contain the high energy absorbing substance 350 however, only absorb the energy. The regions that are made of high energy absorbing substance 350 can be made in different shapes as shown in FIG. 3B (see for example in pattern 3070), or pattern 3080 in FIG. 3C. Of course, a person skilled in the art will easily recognize that the patterns illustrated in FIG. 3B are merely very few representative examples and many other patterns of high absorbing material can be envisioned.

In FIG. 3A, a person skilled in the art will recognize that a beam from the energy source scanned across a portion of the cap 350 and encountering the portion of the cup with a high absorbing substance will deposit its energy in said area according to the rate equation:

Fluence=F₀*Alpha=P/(L Nu Dia)*Alpha

Where F₀ is the energy delivering rate from a scanned source of power P, with a length of scan, L, and scanned frequency Nu, and beam diameter, Dia. The Absorption coefficient Alpha characterizes the high absorbing substance and determines what fraction of the delivered energy is actually absorbed and removed from the beam by the highly absorbing substance.

Said energy will rapidly be conducted downwards to the outside surface of the cap 335 (i.e. the surface in contact with the target material).

The outside surface of the cap 335 is in contact with the target material and will be rapidly heated. Aluminum, for example, may conduct heat across 1 mm distance in 1 ms. Thus a cap with a thickness of for example 100 um may be conducted within 10 us.

Conduction in the lateral dimension is more difficult since it is more difficult to conduct heat in the lateral dimension through a very thin cup. Thermal conduction is accomplished most efficiently through energetic free electron motion and it is clearly more difficult to cram many electrons though a finite narrow passage.

In FIG. 3B we show an additional embodiment using an exemplary cup 3035 with the intermediate material made mostly of a substance of high thermal conductivity, and with an inner surface made with an element that are coated with a high absorbing substance. When the sources beam is scanned across the high absorbing substance its energy is quickly conducted to the outside surface in contact with the target material. On its periphery, the high absorbing substance (HAS)-coated surface may encircled with a thin ring of material of low thermal conductivity. This ring ensures confinement of the thermal effect ONLY to the desired area of HAS coating. Except for the ring of thermal insulator, the entire cap is made of a substance of high thermal conductivity. Thus, when the energy removal substance comes in contact with the inner surface, the entire surface is rapidly cools, thus both quenching the hot thermal area and adjacent regions which may have been effect by thermal energy conduction through the target material itself. This is illustrated in FIG. 3C where the intermediate material 3035 is made of a high conducting substance (HCS), 395. The high absorbing substance area 370, is thermally isolated from the rest of the HCS 395 by the ring of insulator material 390. when the energy source beam is scanned across the cap of intermediate material, 3035, only the area 370 is heated. Heat is confined by the insulating ring to the HAS area 370. However, when the energy removal substance is activated, all the intermediate material area and the target material area in contact with it are cooled.

A complete device and its control box and knobs envisioned by a preferred embodiment of the present invention are illustrated in FIG. 4.

A typical preferred embodiment for a device 4010 consists of five components: a handpiece assembly, a connector cable, power cord, power supply console, and the footswitch.

Within the handpiece assembly are several sub-components:

Diode laser4 4020: Emits laser radiation towards the scanning mirrors.

Thermoelectric cooler 4030: Cools the diode laser, 4020 to prevent overheating.

Photodiode detector 4040: The photodiode detector is used to measure total output power. It compares the amount of photons detected from 4% of the reflected power from the beam splitter to the power output determined by the preset modes.

Scanning unit 4050: The sub-components of the scanning unit consist of mirrors, a lens, and a beam splitter. The mirrors are used to scan the laser emission across a section of the target. The window in the scanning unit is used as a beam splitter where it reflects 4% of the total output beam towards the photodiode detector 4040. This allows for 96% transmission of total energy towards the target tissue. A detailed picture of the scanning unit can be seen in FIG. 4.

Coolant ejector 4060: The coolant ejector sprays the contents in the coolant reservoir 4070 onto the target tissue. The rate and duration at which the coolant is ejected is predetermined by the different preset modes. The ejector sprays once every three scanned lines for a period time period determined by the mode that the device is operating in.

Coolant reservoir 4070: The coolant reservoir houses the coolant that is used by the coolant ejector, 4060.

Focus guard 4080: The focus guard is mounted on the outside of the handpiece. Its length is used to determine to optimal distance from the end of the handpiece to the target tissue. Resting the focus guard on the target tissue does this.

Manual shutter, 4090: The manual shutter is used to prevent accidental firing of the laser. In its open position, it allows transmission of the laser beam to the target tissue. In its closed position, it allows no transmission of laser radiation outside of the handpiece.

The connector cable 4100 is used to connect the handpiece to the power supply console 4110.

The power supply console 4110 contains the control panel, 4120 and houses the power supply 4140. The power supply console is plugged into wall via the power cord 4150. The control panel 4120 is used for turning the device on or off, selecting one of the presets, and displays the system status. The control panel is better illustrated and described in FIG. 5.

The footswitch 4130 is used to operate the device. When depressed, power is supplied to the handpiece that allows the laser diode and all systems within the handpiece assembly to operate.

An exemplary device control box is shown in FIG. 5. The components of such an exemplary device control box are as follows. The push button 510 represent an exemplary present button which, using an exemplary, preprogrammed microprocessor, allow the user to select a predetermined set of scanning, energy source power, and coolant parameters. Four exemplary preset buttons are shown in this example. Above the present buttons 510 an exemplary light emitting diodes (LED) 500 is shown. Such LEDs indicate which settings have been selected. An on/off button is shown by 520. The LED shown by 540 is a standby indicator that lights up when the device is idle. The LED shown by 550 indicates that the energy source is on and interaction with the intermediate and target material is occurring. The LED illustrated by 530 lights up whenever the energy removal components are activated.

As FIG. 6 shows, one preferred embodiment envisioned utilizing a diode laser 120 as an exemplary energy source that is mounted on an thermoelectric cooler. A collimating lens 130 ensures a collimated beam, which then passes through the two scanner mirrors. From the scanner the beam is directed to a focusing lens 140 which directs the laser beam energy to the target tissue. Before emerging from the handpiece a small portion of the beam is directed by the beam splitter 205 to a photodiode detector. The photodiode detector 200 ensures that the system is operated at a proper power level. The coolant reservoir 160 is also mounted within the handpiece. The coolant is ejected via the ejector 170, and directed towards the treated target tissue area. The focus guard 225, ensures proper positioning of the handpiece with respect to the target skin tissue. The manual shutter, 240, prevents accidental firing of the laser. Another possible embodiment makes use of a broadband light source as the source of energy (for example, a halogen lamp or a xenon lamp). Of course, in general, this invention contemplates many energy sources as a possible source.

The laser beam is emitted from the diode laser as a collimated beam, and is reflected from the first scanning mirror, 140, onto the second scanning mirror, 150. From there, the beam passes through the focusing lens, 190. Once the beam reaches the beam splitter 205, 4% of the laser output power 210, is reflected by the beam splitter 205, to the photodiode detector 200, which is used to measure the total amount of power emitted by the diode laser. The photodiode detector 200, ensures that the system is operated at a proper power level. The remaining 96% of the laser output passes through the beam splitter and focuses onto the target tissue.

The coolant reservoir 160, is also mounted within the handpiece. The coolant is elected via the ejector and directed towards the treated target tissue area 180. A manual shutter 240 prevents accidental firing of the laser. The focus guard 225, acts as the positioning device that allows the user to properly determine the optimal distance to the target tissue that will allow for optimal performance of the device.

If the manual shutter 240 is not displaced, the shutter will block all transmission of the beam. Only when the shutter is disengaged from its normal position will the beam be transmitted out of the handpiece and onto the target tissue.

FIGS. 7A through 7F illustrate several other preferred embodiment of the present invention including the patterns of good heat conductors on the intermediate material.

FIG. 7A shows a high thermal conductor ‘heaters’ 720 which comprises part of the intermediate material (which we sometimes also designate at “caps”) and the entire cap structure. The High thermal conductors heaters are separated by the insulator margin 750. Only the HTC 720, are heated due to the High absorbing substance HAS 730, deposited in one end. Heat is conducted throughout the heaters 720 in the direction of the arrows 740. The HASs 730 is heated by the STATIONARY elongated BEAM 710. Thus, FIG. 7A illustrates the high thermally conducting ‘heaters’ 720. The heaters, 720 are covered with the high absorbing substance (HAS) 730. The beam 710 is elongated and its long axis is aligned with that of the long axis of the heaters. The beam is then scanned across the intermediate material and get absorbed every time it crosses each one of the HAS 730 covered heater 720. The rest of the intermediate material 760 is a conductor as well. Heat does not flow from the heaters to the rest of the intermediate material because it is insulated from the rest of the intermediate material conductor by the insulator brackets (750). Upon a predetermined timing an energy removal is activated and quenches the heating of the cell.

FIG. 7B shows the heating process completed by a scanning beam. Here, the entire length of a heater-strip of heaters 720 on the intermediate material 760, is coated by a high absorbing substance 730 and is heated by an elongated beam 710 which is then scanned across the surface and heat each one of the heater as it passes over it.

The entire process can also be made, in another preferred embodiment, by illuminating an entire pattern, as shown in FIG. 7C. The energy source in this case would be a large diameter, large area beam which will irradiate and heat all heater at once. In this preferred embodiment, all the heater-strips 720 on the intermediate material 760, are coated by a high absorbing substance 730 and are heated by a broad beam, 710, which covers and heat all heater-strips at the same time for the duration of time for which the energy source is on.

Both of the above can be made, in another preferred embodiment, using an insulating intermediate substance as the intermediate material 760, said intermediate/insulating material, however, is thin enough to allow quick transfer of heat to the underlying target material. In this case, the High absorbing substance (HAS) is deposited on the insulator—intermediate material—in whatever pattern is desired. The energy source can then be scanned across the absorbers as in 7B, or can be large area as in FIG. 7C. The regions covered with HAS will heat up and transfer their energy to the target material and then, at a predetermined time, the device will activate an energy removing substance, for example a coolant spray, or a cryogen clay, to quench the heating and eliminate some of the pain and control the extent of the interaction and the tissue modification.

FIG. 7D illustrates an exemplary cap 776 comprising of an intermediate material 777 on which a High absorbing substance can be deposited, and a holder 779 that can be attached, via the guiding tube 783, to the handpiece containing the energy source and the energy removing substance.

FIG. 7E shows that cap 776 attached with an intermediate material 777 on which a High absorbing substance can be deposited, with its holder 779, attached, via the guiding tube 783, to the handpiece 781 containing the energy source and the energy removing substance.

In a further preferred embodiment the effect of the energy removal substance interacting directly with the target material or interacting with the intermediate substance absorbing the source's energy can be further controlled through the action of an auxiliary source of energy (or the same energy source activated again for additional periods and acting in coordination with the energy removal substance) in order to mitigate excessive energy removal effect or excessive cooling (which may cause harm to the target material.) For example, in FIG. 7F if the time axis is represented by the line 7035. Then a burst of energy 7005 at time 7006 may be followed by the application of the energy removal substance 7015 at time 7016. Then, in order to control or mitigate the effect of the energy removal substance, the burst 7015 is followed by a second application of a burst of energy pulse 7025 at time 7026. If the target material removal or modification process occur repetitively, then the energy/energy-removal/energy application cycle is repetitive as well as shown by the additional sequences 7040 and 7050 FIG. 7F.

FIG. 8. Another preferred embodiment contemplates “The peeling system” shown in FIG. 8 is made of layers of paper of different color. As FIG. 8 illustrates, one preferred embodiment employed a multileveled high absorbing substance (HAS) system composed of several layers of high absorbing substance. Further, each individual layer may be composed of a different color. The idea is to make use of the fact that

1. The high absorbing substance is consumed during the interaction.

2. The longer the total interaction time is, the deeper the thermal damage and modification to the target material below.

Thus, by employing multilevel system of high absorbing substance, where each layer is composed of a different color, one can calibrate precisely the amount of heat deposited in the tissue. In particular, if one design the thermal conduction properties of the HAS layer, and if one knows how much energy per unit time is required to remove a given thickness of HAS, one can predetermine the amount of thermal energy deposited in the target material.

As FIG. 8 shows, an energy source beam 810 is impinging upon the multi-layer system of high absorbing substance films, 820. The first layer to interact with the beam is layer 830 of a green color, which, for example, can be design so that a given beam/scan parameter set completely removes it in a single path. In doing so, it deposit an amount of X.X W/Cm2 of which y.y W/Cm² is actually deposited in the target material leading to Z1 depth of thermal damage and A1 depth of ablatively removed material.

A second path may be designed to remove the second layer 840 of yellow color, thus depositing Z.Z W/W/Cm² additional power density in the targeted tissue.

One can see that by controlling the interaction parameters and the properties of the high absorbing substance, the manufacturer can allow the user to select a predetermined effect with desired outcome and well known endpoint corresponding to a given color of the exposed layer. Such color selection will tell the user the end treatment end point.

Alternatively in another preferred embodiment, the device may be made of an intermediate material that is capable of absorbing the sources' energy and transmit it to the target material on the other side of the intermediate material in contact with the target tissue. The intermediate target material possesses the property whereby each pass removes some of the intermediate material and exposes a new color or shade. Each such consecutive exposure corresponds do a deeper tissue modification effect.

Alternatively, the intermediate material changes its color with each consecutive application.

Alternatively, the intermediate material is capable of changing its color as a function of cumulative applied.

Alternatively, the intermediate material ability to absorb energy from the source is diminished with consecutive application of energy. Said diminishing ability to absorb is calibrated to allow a known amount of total energy to be absorbed by the intermediate material.

Another embodiment contemplates the intermediate material being able to absorb only a certain amount of energy per time. In other word, its absorbing ability is eliminated after the absorption of a known quantity of energy and then restored after a certain length of recovery time.

In a further embodiment, the above intermediate material is a key to activating the device.

The device cannot be activated and the energy source will not work unless the cup is in place. This is an important safety feature.

It will be appreciated that although preferred embodiments of the present invention are described with respect to the application of energy to skin and the cooling of skin, typically for purposes such as treatment or removal of skin lesions or wrinkles, it is within the scope of the present invention to apply energy to and cool substantially any type of biological tissue. For example, tumors, polyps, hemangiomas, ectasias, arterio-venous malformations, esophageal varices, coronary arteries, hemorrhoids and cellulite deposits are appropriate sites for application of the principles of the present invention. Additionally, non-biological materials may be processed in accordance with some embodiments of the present invention, as described hereinabove. It will be further appreciated that, although preferred embodiments of the present invention are described herein with respect to an electromagnetic energy source, other heating devices are suitable in many applications. For example, these other heating devices may include an ultrasound energy source or a heating element, which is placed in contact with the target area.

Persons skilled in the art will understand that the present invention is not limited to what has been particularly shown and described hereinabove. Rather, the scope of the present invention includes both combinations and sub-combinations of the various features described hereinabove, as well as variations and modifications thereof that are not in the prior art which would occur to persons skilled in the art upon reading the foregoing description.

Additional Embodiments

1. A method for removing or modifying a material, comprising:

applying energy to a material so as to remove or modify a portion of the material; and,

initiating removal of energy from the same material in coordination with the energy deposition phase.

2. The method of claim 1 for removing or modifying a material, comprising:

applying energy to a material so as to remove or modify a portion of the material; and, initiating removal of energy from the same material after the deposition of energy into the material.

3. The method of claim 1 for removing or modifying a material substance, comprising: applying energy to a region of the target material in order to remove or modify a portion of the material in the region; and initiating removal of energy from the same material during the deposition of energy into the material.

4. The method of claim 1 for removing or modifying a material substance, comprising: applying energy to a region of the target material in order to remove or modify a portion of the material in the region; and initiating removal of energy from the material before the deposition of energy into the material.

5. The method of claim 1 for removing or modifying a material substance, wherein the material substance comprises the surface region of the material substance and some of the area below the surface.

6. A method according to claim 1, wherein the material to be modified or removed is a region starting at the surface of a target material and extending into the bulk of the material.

7. The method of claim 1 for removing or modifying a material substance, wherein the modified material substance comprises the surface of the skin and some of the epidermis below the surface but no tissue below the epidermal-dermal junction.

8. A method according to claim 1, wherein initiating energy removal from the material comprises thermoelectrically cooling.

9. A method according to claim 1, wherein initiating energy removal from the material comprises circulating a cool gas through the energy-delivering element in contact with the target material.

10. A method according to claim 1, wherein initiating energy removal from the material comprises applying a cryogen spray cooling.

11. A method according to claim 1, wherein initiating energy removal from the material comprises applying chilled airflow following the energy deposition into the material.

12. A method according to claim 1, wherein initiating energy removal from the material comprises applying a water spray in coordination with (before, during or after) the energy deposition into the material.

13. A method according to claim 1, wherein applying energy comprises applying mechanical energy.

14. A method according to claim 1, wherein applying energy comprises applying mechanical energy thorough the use of a sharp blade or an electro-surgical instrument.

15. A method according to claim 1, wherein applying energy comprises applying energy generated by plasma or applying energy thorough the use of plasma-mediated interaction.

16. A method according to claim 1, wherein applying energy comprises applying thermal energy.

17. A method according to claim 1, wherein applying energy comprises applying optical energy.

18. A method according to claim 1, wherein applying energy comprises applying ultrasound energy.

19. A method according to claim 1, wherein applying energy comprises applying microwave energy.

20. A method according to claim 1, wherein applying energy comprises applying electromagnetic energy.

21. A method according to claim 1, wherein applying energy comprises applying X-Ray energy.

22. A method according to claim 1, wherein applying energy comprises applying the energy of a particle beam.

23. A method according to claim 1, wherein applying energy comprises applying energy which is absorbed by an intermediate substance which is in contact with the target material to be modified or removed, said intermediate substance is capable of absorbing said energy, converting it to thermal energy and conducting the thermal energy to the target material.

24. The method of claim 23 wherein the intermediate substance in contact with the target material and capable of absorbing energy, converting it to thermal energy and conducting said thermal energy to the target material, is cooled after transferring at least part of the absorbed energy to the target material.

25. The method of claim 23 wherein the intermediate substance in contact with the target material is capable of substantially shielding the targeted material from photons and electromagnetic energy cannot be transmitted or pass through it.

26. The method of claim 23 wherein the intermediate substance in contact with the target material is capable of substantially preventing direct contact between the targeted material and any high absorbance substance that is deposited on a surface of the intermediate substance or is included in the intermediate substance.

27. The method of claim 23 wherein the target material is exposed only to the energy conducted through the intermediate substance in contact with the target material, said intermediate substance is capable of absorbing the electromagnetic energy, converting it to thermal energy and conducting it to the target material, and said target material is NOT exposed to any other forms of the applied energy which are not thermally conducted through the substance in contact with the target material.

28. The method of claim 23 wherein some of the energy delivered to the target material is partially removed by applying a substance capable of removing energy to the intermediate substance absorbing the source's energy.

29. The method of claim 28 wherein the substance capable of removing energy from the intermediate substance absorbing the source energy and from the target material is a circulating cooling gas circulating through the intermediate substance.

30. The method of claim 28 wherein the substance capable of removing energy from the intermediate substance absorbing the source energy and from the target material is a cryogen circulating through a thermally conducting element in thermal contact with the intermediate substance and thus also in thermal contact with the target material.

31. The method of claim 28 wherein the effect of the substance capable of removing energy from the intermediate substance absorbing the source energy and from the target material is further controlled or mitigated by the additional application of energy in coordination with said substance capable of removing energy from the intermediate material.

32. A device for modifying and removing material comprising: an energy source and an intermediate material, said energy source is capable of imparting energy to said intermediate material; said intermediate material is capable of absorbing said energy and transferring said energy further to the target material to achieve modification or removal of said target material;

33. The device of claim 32 wherein the energy source emits electromagnetic energy and the intermediate material is composed of a thermally conducting material which is also capable of absorbing said source's electromagnetic energy and then conducting it towards the target material.

34. The device of claim 33 wherein in coordination with transferring the source's energy to the target material a substance capable of removing energy from said intermediate material is applied to the intermediate material.

35. The device of claim 32 wherein the intermediate material comprises a metal having one side in contact with said target material, wherein said metal is comprised of a material that has a high thermal conductivity yet is capable of absorbing the energy source's energy;

36. The device of claim 34 wherein the energy-removing substance comprises a cryogen spray applied to the intermediate material.

37. The device of claim 34 wherein the energy-removing substance is a coolant circulating through the intermediate material or circulating through a thermally conducting container in thermal contact with the intermediate material.

38. The device of claim 32 wherein an energy-removing substance is used in coordination with the deposition of energy in the target material.

39. The device of claim 37, wherein said energy-removing substance comprises a flow of carbon dioxide.

40. The device of claim 37, wherein said energy-removing substance comprises a flow of nitrogen.

41. The device of claim 37, wherein said energy-removing substance comprises air.

42. The device of claim 37, wherein said energy-removing substance comprises freon.

43. The device of claim 37, wherein said energy-removing substance comprises an environmentally friendly coolant.

44. The device of claim 33 wherein said thermally conducting intermediate material is formed into a desired shape and pattern and placed with an additional type of intermediate material which is a poor thermal conductor thus allowing target material modification or removal predetermined by the shape of the portion of the intermediate material which is thermal conducting.

45. The device of claim 32, wherein said energy source comprises a laser.

46. The device of claim 32, wherein said energy source comprises an arc lamp.

47. The device of claim 32, wherein said energy source comprises a flash lamp.

48. The device of claim 32, wherein said energy source comprises a xenon lamp.

49. The device of claim 32, wherein said energy source comprises a microwave source.

50. The device of claim 32, wherein said energy source comprises an acoustic energy source. 51. The device of claim 32, wherein said energy source comprises an electric energy source.

52. The device of claim 32, wherein said energy source comprises a current source.

53. The device of claim 32, wherein said energy source comprises a plasma source.

54. A device according to claim 32, wherein directing the energy source to apply the energy comprises generating a beam of energy having a dwell time over a point in the region of less than 25 ms.

55. A device according to claim 32, capable of applying a nutrient to the tissue.

56. A device according to claim 32, wherein the target material comprises a varix.

57. A device according to claim 32, wherein the target material comprises tumorous tissue.

58. A device according to claim 32, wherein the energy source comprises a broadband emission lamp.

59. A device according to claim 32, wherein initiating cooling comprises applying a cooled surface to the target material.

60. A device according to claim 59, wherein a cooled surface can be applied to the target by activating an oscillating material body in contact with the target material, said oscillating material body is in contact with the target material during one phase of its oscillation and is not in contact with the target material during another phase of its oscillation.

61. A device of claim 34, capable of initiating energy removal by applying a coolant to the target material.

62. A device according to claim 61, wherein the applied coolant comprises a liquid and wherein the device is capable of directing a flow of a gas towards a site on the tissue having the liquid coolant applied thereto, so as to increase the rate of evaporation of the liquid coolant.

63. A device for depositing energy into a target material, the device comprises 1) an energy source, 2) an intermediate medium in contact with the target area capable of absorbing said source's energy said intermediate medium also capable of conducting said energy to the target material, 3) a source of material substance, said material substance is capable, upon contact with the intermediate material, of removing at least some energy from said intermediate medium.

64. The device of claim 63 wherein the substance in the intermediate medium is capable of absorbing said source energy is located on the side farther away from the target material

65. The device of claim 63 wherein the substance in the intermediate medium capable of absorbing said source energy is located on the side farther away from the target material and said absorbing substance is consume by the interaction.

66. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance.

67. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors.

68. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponding to different tissue modification effect.

69. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponding to different tissue modification effect, wherein said energy source is a laser source

70. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponding to different tissue modification effect, wherein said energy source is a laser source

71. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponding to different tissue modification effect, wherein said energy source is a laser source which is directed towards a single spot of an the absorbing substance in the intermediate medium capable of absorbing said laser source energy.

72. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponds to different tissue modification effect, wherein said energy source is a laser source which is directed towards a stirring mechanism capable of moving the beam in a predetermined pattern over absorbing substance in the intermediate medium capable of absorbing said laser source energy.

73. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponds to different tissue modification effect, wherein said energy source is a laser source which is directed towards a stirring mechanism capable of moving the beam in a predetermined pattern over absorbing substance in the intermediate medium capable of absorbing said laser source energy, wherein parts of said intermediate substance are capable of conducting said absorbed energy to the target surface and part of said intermediate medium are insulators

74. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponds to different tissue modification effect, wherein said energy source is a laser source which is directed towards a stirring mechanism capable of moving the beam in a predetermined pattern over absorbing substance in the intermediate medium capable of absorbing said laser source energy, wherein parts of said intermediate substance are capable of conducting said absorbed energy to the target surface and parts of said intermediate medium are insulators.

75. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponds to different tissue modification effect, wherein said energy source is a laser source which is directed towards a stirring mechanism capable of moving the beam in a predetermined pattern over absorbing substance in the intermediate medium capable of absorbing said laser source energy, wherein parts of said intermediate substance are capable of conducting said absorbed energy to the target surface and parts of said intermediate medium are insulators, wherein said laser source is of an energy of less then about 1.0 W.

76. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponds to different tissue modification effect, wherein said energy source is a laser source which is directed towards a stirring mechanism capable of moving the beam in a predetermined pattern over absorbing substance in the intermediate medium capable of absorbing said laser source energy, wherein parts of said intermediate substance are capable of conducting said absorbed energy to the target surface and parts of said intermediate medium are insulators, wherein said laser source is of an energy of less then about 0.5 W.

77. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponds to different tissue modification effect, wherein said energy source is a broadband electromagnetic source which is directed towards a stirring mechanism capable of moving the beam in a predetermined pattern over absorbing substance in the intermediate medium capable of absorbing said laser source energy, wherein parts of said intermediate substance are capable of conducting said absorbed energy to the target surface and parts of said intermediate medium are insulators.

78. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponds to different tissue modification effect, wherein said energy source is a an ultrasound source which is directed towards a stirring mechanism capable of moving the beam in a predetermined pattern over absorbing substance in the intermediate medium capable of absorbing said laser source energy, wherein parts of said intermediate substance are capable of conducting said absorbed energy to the target surface and parts of said intermediate medium are insulators.

79. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponds to different tissue modification effect, wherein said energy source is a microwave source which is directed towards a stirring mechanism capable of moving the beam in a predetermined pattern over absorbing substance in the intermediate medium capable of absorbing said laser source energy, wherein parts of said intermediate substance are capable of conducting said absorbed energy to the target surface and parts of said intermediate medium are insulator.

80. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponds to different tissue modification effect, wherein said energy source is a and X-Ray source which is directed towards a stirring mechanism capable of moving the beam in a predetermined pattern over absorbing substance in the intermediate medium capable of absorbing said laser source energy, wherein parts of said intermediate substance are capable of conducting said absorbed energy to the target surface and parts of said intermediate medium are insulators

81. The device of claim 63 wherein the substance in the intermediate medium capable of absorbing said source energy is located on the side farther away from the target material and said absorbing substance is gradually consume by the interaction thus gradually exposing deeper and deeper layers of the absorbing substance, said different layers possess different colors, and said different layers' color corresponds to different tissue modification effect, wherein said energy source is a and X-Ray source which is directed towards a stirring mechanism capable of moving the beam in a predetermined pattern over absorbing substance in the intermediate medium capable of absorbing said laser source energy, wherein parts of said intermediate substance are capable of conducting said absorbed energy to the target surface and parts of said intermediate medium are insulators and wherein said intermediate substance is located within a attachment capable of being attached to a second compartment containing the energy source, said energy source is unable to operate or emit any energy without said the attachment being properly connected to the second compartment containing said energy source.

82. The device of claim 63 wherein said intermediate substance is located within a attachment capable of being attached to a second compartment containing the energy source, said energy source is unable to operate or emit any energy without said the attachment being properly connected to the second compartment containing said energy source

83. The device of claim 63 wherein said intermediate substance is located within a attachment capable of being connected to a second compartment containing the energy source, said energy source is unable to operate or emit any energy without said the attachment being properly connected to the second compartment containing said energy source, thus ensuring that no radiation is emitted unless an attached cup connects, absorbs and contain said emitted radiation.

84. The device of claim 63, wherein said intermediate substance is a good conductor covered at desired locations with a highly absorbing substance, said intermediate substance is in contact with the skin.

85. The device of claim 63, wherein said intermediate substance is a good conductor covered at desired locations with a highly absorbing substance, said intermediate substance is in contact with the skin, said intermediate substance is capable of heating the skin with energy from a laser source, said intermediate substance is then capable of cooling the skin when a coolant is applied to it.

86. The device of claim 63, wherein said intermediate substance is a good conductor covered at desired locations with a highly absorbing substance, said intermediate substance is in contact with the skin, said intermediate substance is capable of heating the skin with energy from a laser source, said intermediate substance is then capable of cooling the skin when a coolant is applied to it, thus allowing thermal modulation of the targeted skin

87. The device of claim 63, wherein said intermediate substance is a good conductor covered at desired locations with a highly absorbing substance, said intermediate substance is in contact with the skin, said intermediate substance is capable of heating the skin with energy from a laser source, said intermediate substance is then capable of cooling the skin when a coolant is applied to it, thus allowing thermal modulation of the targeted skin, said good conductor intermediate medium allowing the transfer of thermal energy from the location of the highly absorbing substance thought the good conductor intermediate material so the entire intermediate material is heated uniformly thus eliminating the need to steer the beam of energy. This eliminates the need for scanners

88. A devise for modifying the condition of a material, comprising:

A source of optical energy for irradiating an intermediate material with electromagnetic radiation said intermediate material is substantially a strong absorber of said electromagnetic radiation;

cooling element coupled to said source of energy for cooling the surface of the target material immediately after the irradiation period, said cooling means comprising: a thermally conducting intermediate material having one side in contact with the target material, wherein said conducting intermediate material is comprised of a material that is opaque to the source's optical energy and has a high thermal conductivity; and means for circulating a cooling gas to contact with said intermediate material.

89. The device of claim 88, wherein said irradiating means comprises an arc lamp.

90. The device of claim 88, wherein said cooling gas comprises freon.

91. The device of claim 88, wherein said cooling gas comprises carbon dioxide.

92. The device of claim 88, wherein said intermediate material is a metal.

93. The device of claim 88, wherein said intermediate material is a conducting material.

94. The device of claim 88, wherein said intermediate material is a thin non-conducting material.

95. The device of claim 88, wherein said intermediate material is a thin paper.

96. The device of claim 88, wherein said intermediate material is degraded by the interaction.

97. The device of claim 88, wherein said intermediate material is degraded by the interaction at a given target material location and is replaced before use in new location by placing a fresh, unused, segment of intermediate material in contact with the new segment of target material to be modified

98. The device of claim 88, wherein said intermediate material is degraded by the interaction at a given target material location and is replaced before use in new location by placing a fresh, unused, segment of intermediate material in contact with the new segment of target material to be modified, said intermediate material is shaped in the form of a tape which is wrapped around two rollers much like an audiotape or videotape is wrapped around two rollers which allow it to advance.

99. The device of claim 88, wherein said intermediate material is degraded by the interaction at a given target material location and is replaced before use in new location by placing a fresh, unused, segment of intermediate material in contact with the new segment of target material to be modified, said intermediate material is shaped in the form of a tape which is wrapped around two rollers much like an audiotape or videotape is wrapped around two rollers which allow it to advance, said advance is achieved by manual activation of the rollers by the operator.

100. The device of claim 88, wherein said intermediate material is degraded by the interaction at a given target material location and is replaced before use in new location by placing a fresh, unused, segment of intermediate material in contact with the new segment of target material to be modified, said intermediate material is shaped in the form of a tape which is wrapped around two rollers much like an audiotape or videotape is wrapped around two rollers which allow it to advance, said advance is achieved by an automatic activation of the rollers following completion of treatment of each individual site and cessation of action of the energy source and, when appropriate, the energy removal source.

101. The device of claim 88, wherein said intermediate material is degraded by the interaction at a given target material location and is replaced before use in new location by placing a fresh, unused, segment of intermediate material in contact with the new segment of target material to be modified, said intermediate material is placed at equal distance form the center of the disc shaped like a circular plate similar in shape to a compact disk.

102. The device of claim 88, wherein said intermediate material is degraded by the interaction at a given target material location and is replaced before use in new location by placing a fresh, unused, segment of intermediate material in contact with the new segment of target material to be modified, said intermediate material is placed at equal distance form the center of the disc shaped like a circular plate similar in shape to a compact disk, said disk is allowed to advance by the operator using manual activation of the disk spinning mechanism so that a new, fresh and unused intermediate material is placed in contact with the target material before treatment of each new target material location.

103. The device of claim 88, wherein said intermediate material is degraded by the interaction at a given target material location and is replaced before use in new location by placing a fresh, unused, segment of intermediate material in contact with the new segment of target material to be modified, said intermediate material is placed at equal distance form the center of the disc shaped like a circular plate similar in shape to a compact disk, said disk is advanced by automatic activation of the disk spinning mechanism so that a new, fresh and unused intermediate material is placed in contact with the target material before treatment of each new target material location.

104. The device of claim 88, wherein said intermediate material is degraded by the interaction at a given target material location and is replaced before use in new location by placing a fresh, unused, segment of intermediate material in contact with the new segment of target material to be modified, said intermediate material is shaped in the form of cap that can be attached, snapped on to, screwed on to, or attached by any other method to the rest of the device, be brought into contact with the skin and be replaced after each use at a given target material location.

105. The device of claim 88, wherein said intermediate material is degraded by the interaction at a given target material location and is replaced before use in new location by placing a fresh, unused, segment of intermediate material in contact with the new segment of target material to be modified, said intermediate material is shaped in the form of cap that can be attached, snapped on to, screwed on to, or attached by any other method to the rest of the device, be brought into contact with the skin and be replaced after several uses at a given target material location.

106. A devise for modifying the condition of a target material, comprising:

A source of energy embedded in an intermediate material and said intermediate material is capable of transferring thermal energy to and from the target material,

Energy removal component capable of removing energy from the target material.

107. The device of claim 106, wherein said source of energy comprises heater

108. The device of claim 106, wherein said source of energy comprises an electric heater

109. The device of claim 106 wherein said source of energy is continuously on and continuously transferring energy to the target material

110. The device of claim 106 wherein said source of energy is continuously on and continuously transferring energy to the target material while said energy removal component is activated periodically to removed energy from both the intermediate material and the target material

111. The device of claim 106 wherein said source of energy is continuously on and continuously transferring energy to the target material while said energy removal component is activated periodically to removed energy from both the intermediate material and the target material, said activation of energy removal component is preprogrammed to achieve a desired heating and cooling cycle

112. The device of claim 106 wherein said source of energy is continuously on and continuously transferring energy to the target material while said energy removal component is activated periodically to removed energy from both the intermediate material and the target material, said activation of energy removal component is controlled by determining the surfaces temperature.

113. The device of claim 106 wherein said source of energy is continuously on and continuously transferring energy to the target material while said energy removal component is activated periodically to removed energy from both the intermediate material and the target material, said activation of energy removal component is controlled by determining the surfaces temperature and using a component of the device capable of feeding back the target material thermal characteristics component so that said feedback information control the activation of the energy removal component.

114. The device of claim 106 wherein said source of energy is an electric heater capable of conducting thermal energy and the source of cooling is a cryogen cooling spray

115. The device of claim 106 wherein said source of energy is continuously on and continuously transferring energy to the target material while said energy removal component is activated periodically to removed energy from both the intermediate material and the target material, said activation of energy removal component is preprogrammed to achieve a desired heating and cooling cycle and wherein the source of energy is an electric heater and the source of cooling is a cryogen cooling spray

116. The device of claim 106, wherein said source of energy is a heater capable of rapidly being turned off or on and the component capable of energy removal is capable of rapidly removing the energy from the intermediate material.

117. The device of claim 106, wherein said source of energy is a heater capable of being rapidly turned off or on and the component capable of energy removal is a capable of rapidly removing the energy directly from the target material.

118. A system for removing or modifying a material substance, comprising:

an energy source capable of applying energy to a region of the target material, so as to remove or modify a portion of the material in the region; and

An energy removal element capable of removal of residual energy subsequent to the material removal or modification of the target material.

119. The system of claim 118 for removing or modifying a material substance, further comprising of an element capable of synchronizing the action of the element responsible for energy deposition in the target material to the action of the element responsible for energy removal from the target material.

120. The System of claim 118 wherein the energy source is a laser

121. The System of claim 118 wherein the energy source is an electric heater

122. The System of claim 118 wherein the energy source is a thermoelectric cooler

123. The System of claim 118 wherein the energy source is a source of electromagnetic radiation

124. The System of claim 118 wherein the energy source is a source of acoustical energy

125. The System of claim 118 wherein the energy source is a source of mechanical energy

126. The System of claim 118 wherein the energy source is a hot steam

127. The System of claim 118 wherein the energy source is and energized fluid

128. The System of claim 118 wherein the energy source is an energized liquid

129. The System of claim 118 wherein the energy source is an energized solid

130. The System of claim 118 wherein the energy source is a light source

131. The System of claim 118 wherein the energy source is a microwave radiation

132. The System of claim 118 wherein the energy source is a source of nuclear energy

133. The System of claim 118 wherein the energy source is a source of chemical energy

134. The System of claim 118 wherein the energy removal source is a cold liquid

135. The System of claim 118 wherein the energy removal source is a cold fluid

136. The System of claim 118 wherein the energy removal source is a rapidly evaporating gas

137. The System of claim 118 wherein the energy removal source is a thermo eclectic cooler

138. The System of claim 118 wherein the energy removal source is a gas such as freon or freon-like gas or CO2 or any other rapidly evaporating gas.

139. The System of claim 118 wherein the energy removal source is a cool air

140. A DEVICE FOR modifying material at both the surface level AND deeper into the material bulk, the device comprises of (See FIG. 10A) An energy source 1005, said energy 1007 can penetrate the martial and GRADUALLY (with respect to depth) can be deposited within it, yet said energy is also being capable of being absorbed by a substance capable of high absorption of said sources energy, said high absorbing substance (HAS) 1020, is place at some location on the surface of an intermediate material medium (IMM) or a window 1010, the surface which is closer to the energy source and further from the material to be modified 1030. Part of the energy 1047 is absorbed by the HAS on the surface of the window or intermediate material medium (IMM), and part of the energy 1057 penetrates into the material to be modified and there it is scattered and propagates inward 1057. As the energy 1057 (for example, optical energy) is penetrating inward into the material, it is being gradually absorbed in the material and converted to heat and thermal energy, interacting with the material to be modified and modifying and conditioning it. At the same time the portion of the energy from the energy source encountering the HAS 1047 is substantially completely absorbed by the HAS and then thermally conducted 1067 to the surface of the material to be modified below it. If the IMM or window is made of a thermally conducting substance, for example, sapphire, then said thermal energy diffusion out of the HAS region, also diffuses laterally and substantially creating a uniform surface layer (blanket) of thermal energy over the modified material surface, said blanket of thermal energy diffuses and modifies the surface of the modified material.

Said modification of surface region may be accomplished more rapidly and with higher temperatures, since the entire absorbed surface may be concentrated in a narrow surface area.

After a predetermined length of time (determined by the operator/designer to be sufficient to generate enough thermal energy and enough thermal energy penetration and enough thermal energy interaction with the material to be modified, an energy removal source is activated to discharge and energy removal substance, said energy removal substance remove the energy from the HAS, and from the IMM. The removed energy allow the IMM temperature to drop and the new thermal gradient thus generated cause thermal energy flow BACK from the material to be modified to the IMM, thus removing material from the material to be modified, and quenching the interaction.

Said IMM may also be hollow so that energy-removing-substance may circulate through it, thus achieving more rapid and efficient energy removal from the material to be modified. Alternatively, the energy removal substance may be sprayed on the IMM surface closer to the energy source and further from the material to be modified. Alternatively, the energy removal substance may be applied directly to the surface fo the material to be modified, thus allowing a very efficient energy removal.

141. The device of claim 140 wherein the intermediate material substance is composed of (See FIG. 101A) partially reflecting substance 1110 so that partial perforation of the HAS film or HAS cap or HAS cartridge results in detectable drop In the backscattered radiation level. This backscattered light is detected by a detector substantially feedback the signal to the source, automatically stopping the source from emitting energy if said signal level drops below a predetermined level. See FIG. 101A Below.

142. The device of claim 141 wherein the intermediate material substance is highly absorbing and not thermally conducting but a few reflecting substance element are dispersed on the surface of the intermediate material substance facing the energy source. The reflected energy is then detected by a photo-detector. If said reflected energy drops below a certain level, thus indicating possible perforation of the HAS cap, the energy source is immediately disabled. This will ensure that no energy comes out of the cap if said HAS cap is perforated or otherwise allows energy leakage.

143. A method and a device for controlling the distribution of energy in a target material where in the surface is heated by energy of the source absorbed by the high absorbing substance (1100 in FIG. 103A) is diffused to the surface of the material to be modified 1105. This source energy absorption at the surface by the high absorbing substance (HAS) is then converted to thermal energy 1120 and diffuses as thermal energy 1120 into the material to be modified material in the direction of the arrows 1115. On the other hand, some of the source energy is allowed to pass through the intermediate material substance 1005.

FIG. 104A shows what happens to the source's energy that is allowed to pass through the windows 1110. This direct source energy 1130 passing thorough the windows 1110, is then scattered as it propagate through the material to be modified 1105, essentially creating source energy diffusion (or optical energy diffusion if the source energy is optical in an exemplary case). The diffused source energy 1130 penetrates much deeper into the target material 1105, and then gradually is absorbed by the target material thus creating much deeper thermal (or even mechanical or chemical effects). The combination of low lying diffusion thermal energy and deep-penetrating source energy allow the operator to tailor and engineer any spatial energy distribution desired.

Finally, a method and a device for creating thermal (as well as source energy/thermal energy effect at the targeted material 1105 (see FIG. 105A), while allowing rapid energy removal at the end of the energy deposition cycle. A highly conducting intermediate material 1205 which is also substantially transparent to the source energy is coated with HAS 1100 at the surface closest to the target material. The absorbed source energy is then converted to thermal energy 1115 which then diffuses into the target material 1105. At the desired instance in time, a cooling agent 1220 is released and allowed to circulate within the intermediate material 1205 thus removing energy from the surface of the target material 1105 minimizing discomfort and reducing residual damage. Subsequently, the cooling agent 1220 is removed and the intermediate material is ready for further use when the energy source is activated again.

Figure xxx-yyy

Yuv bibi bibi NEW MOTHOD/device added!!______-→

A DEVICE FOR modifying material at both the surface level AND deeper into the material bulk, the device comprises of

An energy source, said energy can penetrate the martial and GRADUALLY (with respect to depth) can be deposited within it, yet said energy is also being capable of being absorbed by a substance=high absorbing substance (HAS), said HAS, is place at some location on the surface of an intermidiate material medium (IMM) or a window (the surface which is closer to the energy source and further from the material to be modified). Part of the energy is absorbed by the HAS on the surface of the window or intermediate material medium (IMM), and part of the energy penetrates into the material to be modified and there it is scattered and propagates inward. As the energy (for example, optical energy) is penetrating inward into the material, it is being gradually absorbed in the material and converted to heat and thermal energy, interacting with the material to be modified and modifying and conditioning it. At the same time the portion of the energy from the energy source encountering the HAS is substantially completely absorbed by the HAS and then thermally diffuses to the surface of the material to be modified below it. If the IMM or window is made of a thermally conducting substance, for example, saphire, then said thermal energy diffusion out of the HAS region, also diffuses laterally and substantially creating a uniform surface layer (blanket) of thermal energy over the modified material surface, said blanket of thermal energy diffuses and modifies the surface of the modified material.

Said modification of surface region may be accomplished more rapidly and with higher temperatures, since the entire absorbed surface may be concentrated in a narrow surface area.

After a predetermined length of time (determined by the operator/designer to be sufficinet to generate enough thermal energy and enough thermal energy penteration and enough thermal energy interaction with the material to be modified, an energy removal source is activated to discharge and energy removal substance, said energy removal substance remove the energy from the HAS, and from the IMM. The removed energy allow the IMM termperature to drop and the new thermal gradient thus generated cause thermal energy flow BACK from the matieral to be modified to the IMM, thus removing material from the material to be modified, and quenching the interaction.

Said IMM may also be hollow so that energy-removing-substance may circulate thorugh it, thus achieving more rapid and efificent energy removal from the material to be modified. Alternatively, the energy removal substance may be sprayed on the IMM surface closer to the enrgy source and further from the material to be modifed. Alternatively, the energy removal substance may be applied directly to the surface fo the material to be modified, thus allowing a very efficient energy removal.

A device and a method for removing or modifying a material substance, comprising: applying energy to an intermediate material, said intermediate material subsequently delivery said energy to the target material. The device and the method also utilize active energy removal schemes in order to minimize pain, and/or protect some tissue and or control tissue modification and removal effects.

### PREFERRED EMBODIMENT—LAYERED INTERMEDIATE MATERIAL/HIGH ABSORBING SUBSTANCE

FIG. 8 shows another preferred embodiment of the present invention. In this embodiment, a grid 810 made of a rigid material is attached to a film of a substance of high absorption 820 to form an interface between the impinging source's energy and the target material. The film of substance of high absorption 820 is in contact with the target material, while the grid 810 is facing the energy source. The grid 810 can be made of aluminum or any other substance. Preferably it is made of material that does not absorb the source energy very well. The energy from the source, passes through the openings in the grid and is then absorbed by the film of high absorbing substance 820 and is then transferred to the target tissue. The energy is either not absorbed well by the grid or if absorbed, is not sufficiently damage the grid. The grid 810 may also serves as a active cooling to cool the target material and the high absorbing substance film. The grid 810 may, for example, function as a thermoelectric cooler where each line on the grid is triggered at a different time (for example, cooling can be triggered so that cooling a line on the grid is triggered to follow an energy scan along or in the vicinity of that line. This phased cooling can assure efficient interaction at, for example, the region parallel to grid line 840 while the region parallel to the area 840 is being infused with energy from the energy source. One exemplary way to prepare such a grid 810 is to use a metallic thin sheet cut out to the desired dimensions, create a perforation pattern in said thin sheet of metal and then, attach the film of high absorbing substance to the sheet of metal.

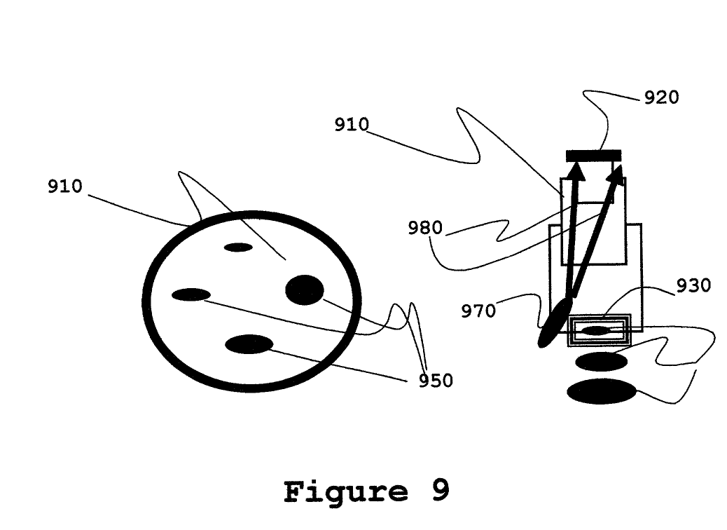

In another prefered embodiment, shown in FIG. 9, an apparatus is contemplated by the invention to allow different focusing lenses to create different spot sizes at the high abosrbing substance. In this embodiment, a telescopic attachment 910 to which a high absorbing substance intermediate material cap 920, is capable of being extended or folded to the desired length from a position, 930 to which a corresponding lens can be attached. The Operator can thus insert a variety of lenses with different focal lengths (thereby creating a range of target effects) and adjust the telescopic attachment 910 to the corresponding proper length. The lenses 950 can either be inserted one by one to a lens holder position 930, or they can be all mounting on an exemplary revolving carrousel or drum 960, said revolving carrousel or drum 960 is placed at the lens holding position 930. The operator can then rotate the revolving carousel or drum to the position where the desire lens is engaged and adjust the telescopic attachment 910 to the proper position. Of course the entire process can be automated and driven by a motor so that the operator can select the target or tissue effect he wish, the drum is automatically rotated to the proper position with the proper lens selection, and the telescopic attachment 910 is automatically adjusted by motor to the appropriate extended length. Also shown in FIG. 9 is an optional energy removal system 970. Such energy removal system can consist, for example, of a coolant reservoir and ejection control, 970, which, for example, on demand, allow an exemplary jet of coolant spray 980 to discharge and be directed toward the intermediate material medium 920 with the high absorbing substance. The cooling of the intermediate material medium also remove energy from the target material and thus the cooling of the both the intermediate material as well as the cooling of the target material is achieved.

BIBI BIBI BIBI BIBI

What is claimed is:

1. A method for the modification of a layer of a tissue comprising the steps of:
applying to a section of the tissue a substance having a high absorption of at least one frequency band of electromagnetic radiation
illuminating the covered section of the targeted tissue with electromagnetic radiation of said at least one frequency band of electromagnetic radiation, said radiation energy is thereby absorbed by the high absorption substance covering the target tissue surface and is thereby converted to thermal energy sufficient to bring about irreversible modification in the skin properties.

2. The method as in claim 1 wherein said high absorption substance is a suspension containing high absorbing particles of a dimension larger then 50 microns.

3. The method as in claim 1 wherein said high absorption substance is a suspension containing high absorbing particles of a dimension larger than 30 microns

4. The method as in claim 1 wherein said high absorption substance is a suspension containing high absorbing particles of a dimension larger than 0.1 microns

5. The method as in claim 1 wherein said high absorption substance is a thin film containing high absorbing particles

6. The process as in claim 5 wherein said high absorption substance is deposited in a thin film containing high absorbing particles of density which assures that at least 60% of the light energy is intercepted and absorbed by the particles.

7. The process as in claim 5 wherein said high absorption substance is deposited in a thin film containing high absorbing particles of density which assures that at least 40% of the light energy is intercepted and absorbed by the particles.

8. The process as in claim 5 wherein said high absorption substance is deposited in a thin film containing high absorbing particles of density which assures that at least 20% of the light energy is intercepted and absorbed by the particles.

9. The process as in claim 1 wherein said high absorption substance is deposited in a thin film containing high absorbing particles of density corresponding to the rate of energy deposition per unit area so that the energy deposited in the skin is sufficient for the removal of at no more than 70% of the epidermis and the energy deposited in the skin allows permanent modification of the skin to a depth of no more then 100 micrometer below said depth of tissue removal.

10. The method of claim 1 where said high absorption substance is a paper containing highly absorbing particles.

11. The method of claim 1 where said high absorption substance is made of agar containing highly absorbing particles

12. The method of claim 1 where said high absorption substance is a mixture containing highly absorbing particles.

13. The method of claim 1 where said high absorption substance is a layer of thermal insulator containing highly absorbing particles.

14. The metho of claim 1 where said high absorption substance is a layer of thermal conductor containing highly absorbing particles

15. The method of claim 1 where said high absorption substance is a metallic layer containing highly absorbing particles.

16. The method of claim 1 wherein said high absorption substance is applied to a film of material on the side facing the energy source and not to the side which is in contact with the skin

17. The method of claim 1 wherein said high absorption substance is applied to a film of material on the side facing the energy source and not to the side which is in contact with the skin, and the film is made of thin layer allowing transmittal of at least some thermal energy to the target material

18. The method of claim 1 wherein said high absorption substance is applied to a film of material on the side facing the energy source and not to the side which is in contact with the target material, and, The film is made of thermally conducting material

19. The method of claim 1 wherein said high absorption substance is mixed with grains of conducting material to form a film of thermally conducting-optically absorbing mix.

20. The method as in claim 5 wherein said high absorption substance is deposited in a thin film containing high absorbing particles of density which assures that at least 80% of the light energy is intercepted and absorbed by the particles.
